(19) United States
(12) Patent Application Publication
(10) Pub. No.: US 2026/0240998 A1
(43) Pub. Date: Aug. 20, 2026

(54) ANTI-BCMA ANTIBODY AND CHIMERIC ANTIGEN RECEPTOR

(71) Applicants: JICHI MEDICAL UNIVERSITY, Tokyo (JP); TAKARA BIO INC., Shiga (JP)

(72) Inventors: Ryosuke UCHIBORI, Shimotsuke-shi, Tochigi (JP); Ken OHMINE, Shimotsuke-shi, Tochigi (JP); Keiya OZAWA, Shimotsuke-shi, Tochigi (JP)

(73) Assignees: JICHI MEDICAL UNIVERSITY, Tokyo (JP); TAKARA BIO INC., Shiga (JP)

(21) Appl. No.: 19/164,755

(22) PCT Filed: Mar. 14, 2024
(86) PCT No.: PCT/JP2024/009945

§ 371 Date: Sep. 12, 2025

(30) Foreign Application Priority Data
Mar. 15, 2023 (JP) 2023-041191

(51) Int. Cl. A61K 40/41; A61K 40/11; A61K 40/33; C07K 16/28

(52) U.S. Cl. CPC A61K 40/41; A61K 40/11; A61K 40/33; C07K 16/2878

(57) ABSTRACT

The present invention provides: a B-cell maturation antigen (BCMA) antibody containing light-chain variable (VL) regions and heavy-chain variable (VH) regions or an antigen-binding fragment of the antibody, wherein the amino acid sequences for complementarity determining region (CDR)1, CDR2 and CDR3 in each of the VL regions and CDR1, CDR2 and CDR3 in each of the VH regions are specified; and a chimeric antigen receptor containing the fragment.

### TECHNICAL FIELD

The present invention relates to antibodies specific for a B-cell maturation antigen (BCMA), antigen-binding fragments thereof, and chimeric antigen receptors comprising said fragments.

### BACKGROUND ART

BCMA, also known as CD269 or TNFRSF17, is a member of tumor necrosis factor receptor superfamily. Ligands for BCMA include a B cell-activating factor (BAFF) and a proliferation-inducing ligand (APRIL). Binding of BCMA to BAFF or APRIL activates NF-kB and induces upregulation of anti-apoptotic Bcl-2 members, such as Bcl-xL or Bcl-2 and Mcl-1. The interaction of BCMA with its ligand regulates various aspects of humoral immunity, B cell growth and differentiation, thereby maintaining a stable equilibrium within the human body environment.

The expression of BCMA is restricted to the B cell lineage. BCMA is expressed on plasmablasts, plasma cells and some mature B cells, and increased during differentiation of peripheral B cells. On the other hand, BCMA is not expressed in most B cells, including naive B cells, memory B cells, germinal center B cells, and other organs. BCMA is also highly expressed in cancer cells. For example, BCMA is detected in cells from patients with multiple myeloma (MM), plasma cell leukemia (PL), and Hodgkin's lymphoma (HL).

In recent years, immunotherapy, particularly adoptive T cell therapy, has shown strong therapeutic efficacy in clinical trials for treating hematological malignancies. T cells genetically modified to express chimeric antigen receptors (CARs) (CAR-T cells) have been developed. CARs comprise an antigen recognition domain and a T cell activation domain. CARs utilize the antigen-binding property of monoclonal antibodies to accomplish targeting without being restricted by MHC. Such non-MHC-restricted antigen recognition circumvents one of tumor immune evasion mechanisms. In this field, there is a strong need for the development of CAR-T cells that target BCMA to treat tumors such as multiple myeloma (Non-Patent Document 1).

### PRIOR ART DOCUMENT

### Non-Patent Document

Non-patent Document 1: Scad J Immunol. 2020 August:92(2): e12910

### SUMMARY OF THE INVENTION

### Problems to be Solved by the Invention

An object of the present invention is to provide a CAR that targets BCMA, an anti-BCMA antibody that targets BCMA, and an antigen-binding fragment thereof.

### Solutions to the Problems

As a result of intensive research to solve the above problems, the present inventors found a novel anti-BCMA antibody that targets BCMA and an antigen-binding fragment thereof, and constructed an effective CAR that targets BCMA. Thus the present invention was completed.

That is, the present invention relates to the followings.

[1] An anti-human B-cell maturation antigen (BCMA) antibody or an antigen-binding fragment thereof, comprising a light chain variable (VL) region and a heavy chain variable (VH) region, wherein the amino acid sequences of complementarity-determining regions (CDR) 1, CDR2, and CDR3 of the VL region and CDR1, CDR2, and CDR3 of the VH region are amino acid sequences set forth in SEQ ID NOs selected from the group consisting of the following (1) to (6):
(1) SEQ ID NOs: 1 to 6,
(2) SEQ ID NOs: 7 to 12,
(3) SEQ ID NOs: 13 to 18,
(4) SEQ ID NOs: 19 to 24,
(5) SEQ ID NOs: 25 to 30, and
(6) SEQ ID NOs: 31 to 36.
   [2] The antibody or antigen-binding fragment thereof according to [1], wherein the amino acid sequences of the VL and VH regions are selected from the group consisting of the following (1) to (6):
(1) SEQ ID NOs: 37 and 38,
(2) SEQ ID NOs: 39 and 40,
(3) SEQ ID NOs: 41 and 42,
(4) SEQ ID NOs: 43 and 44,
(5) SEQ ID NOs: 45 and 46, and
(6) SEQ ID NOs: 47 and 48.
   [3] The antibody or antigen-binding fragment thereof according to [1] or [2], wherein the antibody or antigen-binding fragment thereof is selected from the group consisting of IgG, Fab, Fab′, F(ab′)2, Fv, scFv, and single domain antibody (dAB).
   [4] The antigen-binding fragment according to [3], wherein the antigen-binding fragment is an scFv.
   [5] A nucleic acid encoding the antibody or antigen-binding fragment thereof according to any one of [1] to [4].
   [6] A chimeric antigen receptor (CAR) comprising (i) an antigen-binding domain, (ii) a transmembrane domain, and (iii) an intracellular domain, wherein the antigen-binding domain comprises the antigen-binding fragment according to any one of [1] to [4].
   [7] The CAR according to [6], wherein the intracellular domain comprises a costimulatory domain and an activation domain.
   [8] The CAR according to [7], wherein the costimulatory domain comprises an intracellular fragment of CD28.
   [9] The CAR according to [7] or [8], wherein the activation domain comprises an intracellular fragment of CD3ζ.
   [10] A nucleic acid encoding the CAR according to any one of [6] to [9].
   [11] A vector comprising the nucleic acid encoding the antibody or antigen-binding fragment thereof according to [5] or the nucleic acid encoding the CAR according to [10].
   [12] A cell comprising the nucleic acid encoding the antibody or antigen-binding fragment thereof according to [5] or the nucleic acid encoding the CAR according to [10].
   [13] The cell according to [12], wherein the cell is a T cell.
   [14] A pharmaceutical composition comprising the cells according to [13] as an active ingredient.
   [15] A method for producing a cell expressing a CAR, the method comprising transducing a cell with the nucleic acid encoding the antibody or antigen-binding fragment thereof according to [5], the nucleic acid encoding the CAR according to [10], or the vector according to [11].

### Effects of the Invention

The present invention provides a BCMA-specific antibody and its antigen-binding fragment, as well as a chimeric antigen receptor (CAR) useful in the field of gene-modified immune cell therapy targeting BCMA, a nucleic acid encoding the CAR, a vector containing the nucleic acid, a cell expressing the CAR, a method for producing the cell, a pharmaceutical composition containing the cell as an active ingredient, and a method for preventing (including prevention of recurrence; the same applies hereinafter) or treating a disease, which comprises administering the pharmaceutical composition to a subject. Cells expressing the CAR of the present invention have high killing ability against cells expressing BCMA, and are expected to produce a high therapeutic effect.

### BRIEF DESCRIPTION OF THE DRAWINGS

FIG. 1 shows comparison among the amino acid sequences of anti-BCMA antibodies 1 to 6.

FIG. 2 shows the structure of BCMA-CAR.

FIG. 3 shows the cell signaling intensity of BCMA-CAR-expressing cells.

FIG. 4 shows comparison of linker lengths of anti-BCMA antibodies.

FIG. 5 shows measurements of the cytotoxic activity of BCMA-CAR-expressing cells.

FIG. 6 shows IFN-7 production in BCMA-CAR-expressing cells.

FIG. 7 shows in vivo bioluminescence imaging of luciferase activity in tumor cells over time after infusion of BCMA-CAR-expressing cells, and graphs of quantified luminescence intensity.

FIG. 8-1 shows in vivo antitumor effect of BCMA-CAR-expressing cells on cancer-bearing mice.

FIG. 8-2 shows in vivo antitumor effect of BCMA-CAR-expressing cells on cancer-bearing mice.

FIG. 8-3 shows in vivo antitumor effect of BCMA-CAR-expressing cells on cancer-bearing mice.

FIG. 9-1 shows suppressing effect of BCMA-CAR-expressing cells on the progression of anemia.

FIG. 9-2 shows suppressing effect of BCMA-CAR-expressing cells on the progression of anemia.

FIG. 10-1 shows inhibitory effect of BCMA-CAR-expressing cell therapy on the progression of bone region.

FIG. 10-2 shows inhibitory effect of BCMA-CAR-expressing cell therapy on the progression of bone region.

### MODE FOR CARRYING OUT THE INVENTION

The present invention is explained in detail below.

### (1) Antibody or Antigen-Binding Fragment Thereof of the Present Invention

The present invention provides an antibody that specifically binds to human BCMA, or a fragment of the antibody that specifically binds to human BCMA. The antibody and fragment thereof comprise the amino acid sequences of complementarity-determining region (CDR) 1, CDR2, and CDR3 of a VL region and CDR1, CDR2, and CDR3 of a VH region, derived from any one selected from the group consisting of anti-B cell maturation antigen (BCMA) antibodies 1 to 6 as shown in Examples described later. The amino acid sequences of the complementarity determining regions CDR1 to CDR3 of the VL region and CDR1 to CDR3 of the VH region are shown in the following (1) to (6).
(1) SEQ ID NOs: 1 to 6,
(2) SEQ ID NOs: 7 to 12,
(3) SEQ ID NOs: 13 to 18,
(4) SEQ ID NOs: 19 to 24,
(5) SEQ ID NOs: 25 to 30, or
(6) SEQ ID NOs: 31 to 36.

In one embodiment of the present invention, the amino acid sequences of the VL and VH regions of the anti-BCMA antibody or antigen-binding fragment thereof of the present invention are selected from the group consisting of the following (1) to (6).
(1) SEQ ID NOs: 37 and 38,
(2) SEQ ID NOs: 39 and 40,
(3) SEQ ID NOs: 41 and 42,
(4) SEQ ID NOs: 43 and 44,
(5) SEQ ID NOs: 45 and 46, and
(6) SEQ ID NOs: 47 and 48.

The SEQ ID NOs of the amino acid sequences of the VL CDRs and VH CDRs of anti-BCMA antibodies 1 to 6 of the present invention, the full-length amino acid sequences of VL and VH, and the base sequences encoding the full-length VL and VH are shown in Table 1 below.

TABLE 1

| | SEQ ID NOs | | | | | | | | | |
|---|---|---|---|---|---|---|---|---|---|---|
| | | | | | | | Full-length amino | | Full-length | |
| | VL | | | VH | | | acid | | nucleic acid | |
| Antibody | CDR1 | CDR2 | CDR3 | CDR1 | CDR2 | CDR3 | VL | VH | VL | VH |
| 1 | 1 | 2 | 3 | 4 | 5 | 6 | 37 | 38 | 49 | 50 |
| 2 | 7 | 8 | 9 | 10 | 11 | 12 | 39 | 40 | 51 | 52 |
| 3 | 13 | 14 | 15 | 16 | 17 | 18 | 41 | 42 | 53 | 54 |
| 4 | 19 | 20 | 21 | 22 | 23 | 24 | 43 | 44 | 55 | 56 |
| 5 | 25 | 26 | 27 | 28 | 29 | 30 | 45 | 46 | 57 | 58 |
| 6 | 31 | 32 | 33 | 34 | 35 | 36 | 47 | 48 | 59 | 60 |

In anti-BCMA antibody 1, which is an example of the antibody of the present invention, the VL CDRs 1 to 3 consist of the amino acid sequences of SEQ ID NOs: 1 to 3, respectively, and the VH CDRs 1 to 3 consist of the amino acid sequences of SEQ ID NOs: 4 to 6, respectively, and for example, the VL and VH regions comprise the amino acids of SEQ ID NOs: 37 and 38, respectively, and for example, the VL and VH regions comprise the nucleic acid sequences of SEQ ID NOs: 49 and 50, respectively.

In anti-BCMA antibody 2, which is an example of the antibody of the present invention, the VL CDRs 1 to 3 consist of the amino acid sequences of SEQ ID NOs: 7 to 9, respectively, and the VH CDRs 1 to 3 consist of the amino acid sequences of SEQ ID NOs: 10 to 12, respectively; and for example, the VL and VH regions comprise the amino acids of SEQ ID NOs: 39 and 40, respectively, and for example, the VL and VH regions comprise the nucleic acid sequences of SEQ ID NOs: 51 and 52, respectively.

In anti-BCMA antibody 3, which is an example of the antibody of the present invention, the VL CDRs 1 to 3 consist of the amino acid sequences of SEQ ID NOs: 13 to 15, respectively, and the VH CDRs 1 to 3 consist of the amino acid sequences of SEQ ID NOs: 16 to 18, respectively; and for example, the VL and VH regions comprise the amino acids of SEQ ID NOs: 41 and 42, respectively, and for example, the VL and VH regions comprise the nucleic acid sequences of SEQ ID NOs: 53 and 54, respectively.

In anti-BCMA antibody 4, which is an example of the antibody of the present invention, the VL CDR1 to CDR3 consist of the amino acid sequences of SEQ ID NOs: 19 to 21, respectively, and the VH CDR1 to CDR3 consist of the amino acid sequences of SEQ ID NOs: 22 to 24, respectively; and for example, the VL and VH regions comprise the amino acids of SEQ ID NOs: 43 and 44, respectively, and for example, the VL and VH regions comprise the nucleic acid sequences of SEQ ID NOs: 55 and 56, respectively.

In anti-BCMA antibody 5, which is an example of the antibody of the present invention, the VL CDR1 to CDR3 consist of the amino acid sequences of SEQ ID NOs: 25 to 27, respectively, and the VH CDR1 to CDR3 consist of the amino acid sequences of SEQ ID NOs: 28 to 30, respectively; and for example, the VL and VH regions comprise the amino acids of SEQ ID NOs: 45 and 46, respectively, and for example, the VL and VH regions comprise the nucleic acid sequences of SEQ ID NOs: 57 and 58, respectively.

In anti-BCMA antibody 6, which is an example of the antibody of the present invention, the VL CDR1 to CDR3 consist of the amino acid sequences of SEQ ID NOs: 31 to 33, respectively, and the VH CDR1 to CDR3 consist of the amino acid sequences of SEQ ID NOs: 34 to 36, respectively; and for example, the VL and VH regions comprise the amino acids of SEQ ID NOs: 47 and 48, respectively, and for example, the VL and VH regions comprise the nucleic acid sequences of SEQ ID NOs: 59 and 60, respectively.

The above anti-BCMA antibodies 1 to 6 may comprise one or more mutations, as long as the antibodies specifically bind to human BCMA. Therefore, further examples of the antibody or antigen-binding fragment thereof of the present invention include antibodies or antigen-binding fragments thereof that have specific binding to human BCMA and comprise a VL region and a VH region comprising amino acid sequences selected from the group consisting of the following (1′) to (6′):
(1′) (i) the amino acid sequence of SEQ ID NO: 37, or a mutated amino acid sequence comprising one or more mutations in the amino acid sequence of SEQ ID NO: 37, and (ii) the amino acid sequence of SEQ ID NO: 38, or a mutated amino acid sequence comprising one or more mutations in the amino acid sequence of SEQ ID NO: 38, wherein at least one of (i) or (ii) is the mutated amino acid sequence;
(2′) (i) the amino acid sequence of SEQ ID NO: 39, or a mutated amino acid sequence comprising one or more mutations in the amino acid sequence of SEQ ID NO: 39, and (ii) the amino acid sequence of SEQ ID NO: 40, or a mutated amino acid sequence comprising one or more mutations in the amino acid sequence of SEQ ID NO: 40, wherein at least one of (i) or (ii) is the mutated amino acid sequence;
(3′) (i) the amino acid sequence of SEQ ID NO: 41, or a mutated amino acid sequence comprising one or more mutations in the amino acid sequence of SEQ ID NO: 41, and (ii) the amino acid sequence of SEQ ID NO: 42, or a mutated amino acid sequence comprising one or more mutations in the amino acid sequence of SEQ ID NO: 42, wherein at least one of (i) or (ii) is the mutated amino acid sequence;
(4′) (i) the amino acid sequence of SEQ ID NO: 43, or a mutated amino acid sequence comprising one or more mutations in the amino acid sequence of SEQ ID NO: 43, and (ii) the amino acid sequence of SEQ ID NO: 44, or a mutated amino acid sequence comprising one or more mutations in the amino acid sequence of SEQ ID NO: 44, wherein at least one of (i) or (ii) is the mutated amino acid sequence;
(5′) (i) the amino acid sequence of SEQ ID NO: 45, or a mutated amino acid sequence comprising one or more mutations in the amino acid sequence of SEQ ID NO: 45, and (ii) the amino acid sequence of SEQ ID NO: 46, or a mutated amino acid sequence comprising one or more mutations in the amino acid sequence of SEQ ID NO: 46, wherein at least one of (i) or (ii) is the mutated amino acid sequence;
(6′) (i) the amino acid sequence of SEQ ID NO: 47, or a mutated amino acid sequence comprising one or more mutations in the amino acid sequence of SEQ ID NO: 47, and (ii) the amino acid sequence of SEQ ID NO: 48, or a mutated amino acid sequence comprising one or more mutations in the amino acid sequence of SEQ ID NO: 48, wherein at least one of (i) or (ii) is the mutated amino acid sequence.

As used herein, the term “mutation” includes deletions, additions, insertions and substitutions of amino acid residues. The one or more mutations may be any combination of deletions, additions, insertions, and substitutions of amino acid residues. The amino acid residue substitution may be either a conservative substitution or a non-conservative substitution, and a conservative substitution is preferred. The conservative substitution is a substitution between amino acid residues having similar properties that does not substantially alter the peptide activity, and is well known to those skilled in the art. For example, substitutions between non-polar (hydrophobic) amino acids, substitutions between polar (neutral) amino acids, substitutions between basic amino acids, and substitutions between acidic amino acids are included. Examples of non-polar (hydrophobic) amino acids include alanine, valine, isoleucine, leucine, proline, tryptophan, phenylalanine, and methionine. Examples of polar (neutral) amino acids include glycine, serine, threonine, tyrosine, glutamine, asparagine, and cysteine. Examples of basic amino acids include arginine, histidine, and lysine. Examples of acidic amino acids include aspartic acid and glutamic acid.

The one or more mutations are preferably in a region other than CDRs. The one or more mutations may be, for example, 1 to 10 mutations, preferably 1 to 9, 8, 7, 6, 5, 4, 3 or 2 mutations, more preferably 1 to 3 mutations, and even more preferably 1 or 2 mutations.

The mutated amino acid sequence is, for example, an amino acid sequence having at least 80%, 85%, 90%, 91%, 92%, 93%, 94%, 95%, 96%, 97%, 98%, or 99% sequence identity to the amino acid sequence of any one of SEQ ID NOs: 37 to 48. As used herein, percent (%) identity is determined by aligning a reference amino acid sequence and a comparative amino acid sequence to maximize % identity. Percent (%) identity can be determined using a method known in the art, for example BLAST, FASTA, or the like.

An “antibody” (Ab) is an immunoglobulin that specifically binds to an antigen and may comprise at least two heavy chains (H chains) and two light chains (L chains) interconnected by disulfide bonds, or their antigen-binding molecules. Each H chain comprises a VH region and a heavy chain constant region. The heavy chain constant region includes three constant domains, namely CH1, CH2 and CH3. Each light chain comprises a VL region and a light chain constant region. The light chain constant region includes one constant domain, namely a CL region. The VH and VL regions can be further subdivided into conserved regions called framework regions (FRs) and hypervariable regions called complementarity-determining regions (CDRs). VH and VL each comprise three CDRs and four FRs, wherein FR1, CDR1, FR2, CDR2, FR3, CDR3, and FR4 are arranged from the amino terminus to the carboxy terminus in this order. The variable regions of the heavy and light chains contain an antigen-binding site that interacts with an antigen.

The term “antibody” includes, for example, monoclonal antibodies, polyclonal antibodies, recombinant antibodies, multispecific antibodies (including bispecific antibodies), human antibodies, humanized antibodies, antibody variants, chimeric antibodies, synthetic antibodies, antibody light chain monomers, antibody heavy chain monomers, antibody light chain dimers, antibody heavy chain dimers, antibody light chain-antibody heavy chain pairs, intrabodies, antibody fusions (antibody conjugates), single chain antibodies (scFv), affibodies, Fab fragments, F(ab′)2 fragments, disulfide-linked Fv (sdFv), anti-idiotypic (anti-Id) antibodies (including, for example, anti-anti-Id antibodies), minibodies, domain antibodies, antibody mimics, and their antigen-binding fragments. The major classes of antibodies are IgA, IgD, IgE, IgG, and IgM. Based on the amino acid sequences of heavy chain constant domains, known are alpha (α), delta (δ), epsilon (ε), gamma (γ), and mu (μ), which give rise to the IgA, IgD, IgE, IgG including IgG1, IgG2, IgG3, and IgG4 subclasses, and IgM class antibodies, respectively. Based on the amino acid sequences of light chain constant domains, for example, subclasses of kappa (κ) or lambda (λ) are known. The antibody of the present invention may be of any class or subclass.

The “antigen-binding fragment” refers to any antigen-binding molecule that contains the antigen-binding portion (e.g., CDRs) of an antibody. The antigen-binding fragment of the present invention comprises CDRs derived from the antibody of the present invention. Examples of the antigen-binding fragment of the present invention include, but not limited to, Fab, Fab′, F(ab′)2, Fv fragments, linear antibodies, scFv, single-domain antibodies, and multispecific antibodies formed from antigen-binding molecules. The antigen-binding fragment of the present invention may be in any of the forms described above. As one embodiment, the antigen-binding fragment of the present invention may be an scFv. The scFv is a heterodimer of VH and VL covalently linked via a linker peptide. The linker peptide is, for example, a peptide of about 10 to about 25 amino acids. The linker peptide is rich in glycine for flexibility, as well as serine or threonine for solubility. The linker may either connect the N-terminus of the VH region to the C-terminus of the VL region, or may connect the C-terminus of the VH region to the N-terminus of the VL region. That is, the linker, VH and VL may be connected in the order of VL-linker-VH or VH-linker-VL from the N-terminus to the C-terminus. Examples of linker sequences useful in the present invention include, but not limited to, the linker sequences of glycine/serine linkers, for example, GGGSGGGSGGGS (SEQ ID NO: 61), Gly4Ser (G4S) linkers such as a (G4S)3 linker (SEQ ID NO: 62: GGGGSGGGGSGGGGS) and a (G4S)4 linker (SEQ ID NO: 63: GGGGSGGGGSGGGGSGGGGS), and GGSSRSSSSGGGGSGGGG (SEQ ID NO: 64). The ScFv retains the specificity of the original immunoglobulin despite the removal of constant regions and the introduction of a linker.

The antibody or antigen-binding fragment thereof of the present invention may have a FR derived from any of antibodies 1 to 6, or may have a FR derived from a different antibody, for example, from an antibody of a different animal species, as long as the antibody or antigen-binding fragment thereof of the present invention retains the ability to bind to human BCMA. The present invention also encompasses antibodies produced by grafting the CDRs provided by the present invention into other FRs by a method well known to those skilled in the art, and antigen-binding fragments of the antibodies. Furthermore, the antibody of the present invention may have a constant region derived from any of antibodies 1 to 6, or may have a constant region derived from another antibody, for example, an antibody from a different animal species.

The binding affinity for the antigen (BCMA) of the antibody of the present invention may be generally represented by a dissociation constant (KD). The affinity may be measured and/or expressed in many ways known in the art, including, but not limited to, an equilibrium dissociation constant (KD) and an equilibrium association constant (KA). KD is calculated from the quotient of koff/kon, while KA is calculated from the quotient of kon/koff. The kon refers to an association rate constant of, for example, an antibody to an antigen. The koff refers to a dissociation constant of, for example, an antibody to an antigen. The kon and koff can be determined by a technique known to those skilled in the art such as BIACORE™ or KinExA.

### (2) Chimeric Antigen Receptor (CAR)

The present invention includes a chimeric antigen receptor (CAR) comprising (i) an antigen-binding domain, (ii) a transmembrane domain, and (iii) an intracellular domain, wherein the antigen-binding domain comprises the anti-BCMA antibody or antigen-binding fragment thereof of the present invention. The CAR of the present invention is a recombinant receptor. A nucleic acid encoding the receptor is readily introduced into immune cells including T cells by a technique known in the art, and expressed in the cells. The immune cells recognize BCMA by the CAR, and at the same time, are activated via the CAR bound to BCMA. The activated immune cells attack and destroy cells expressing BCMA. When BCMA is present on tumor cells, immune cells expressing CAR can damage and kill the tumor cells.

### (i) Antigen-Binding Domain

The antigen-binding domain contained in the CAR of the present invention is a polypeptide having six CDRs derived from any of the anti-BCMA antibodies 1 to 6 of the present invention, and is preferably an antigen-binding fragment, for example one or more scFvs, of any of antibody 1 to 6. The antigen-binding domain, which may also include a spacer domain or a leader peptide as described later, is sometimes referred to as the extracellular domain.

In one embodiment of the present invention, the CAR of the present invention may comprise a hinge domain or a spacer domain between the antigen-binding domain and the transmembrane domain. The terms “hinge domain” and “spacer domain” are used interchangeably herein. This domain contains up to 300 amino acids, preferably 10-100 amino acids, most preferably 25-50 amino acids. The spacer domain is preferably a sequence that promotes binding between the CAR and an antigen and enhances intracellular signal transduction. Examples of amino acids that are expected to promote the binding include cysteine, charged amino acids, and amino acids such as serine or threonine in potential glycosylation sites, and such amino acids can be used as amino acids that constitute the spacer domain. The spacer domain can be all or a portion of the hinge region, amino acids 118 to 178 of CD8a (NCBI RefSeq: NP_001759.3), amino acids 135 to 195 of CD80 (GenBank: AAA35664.1), amino acids 315 to 396 of CD4 (NCBI RefSeq: NP_000607.1), or amino acids 137 to 152 of CD28 (NCBI RefSeq: NP_006130.1). Furthermore, a part of the constant region of an antibody H chain or L chain (CH1 region or CL region) can also be used as the spacer domain. Furthermore, the spacer domain may be an artificially designed sequence. The CAR of the present invention can be designed to form a multimer, particularly a dimer. For example, the CAR is multimerized (dimerized) by inserting cysteines into the spacer domain and/or transmembrane domain.

In one embodiment of the present invention, the CAR of the present invention may further comprise a leader peptide (also referred to herein as a “signal peptide” or “leader sequence”) at its N-terminus. The polynucleotide and polypeptide sequences of leader peptides are known in the art. For example, the leader peptide of the GM-CSF receptor can be used.

### (ii) Transmembrane Domain

The CAR of the present invention comprises a transmembrane domain that directly or indirectly links the antigen-binding domain and the intracellular domain. Furthermore, the CAR of the present invention may optionally contain a linker sequence or a spacer sequence between these domains. The transmembrane domain may be derived from a naturally occurring polypeptide or may be artificially designed. The transmembrane domain derived from a naturally occurring polypeptide can be obtained from any membrane-bound or transmembrane protein. For example, the transmembrane domains of the α and β chains of the T cell receptor, the CD3ζ chain, CD28, CD3ε, CD45, CD4, CD5, CD8, CD9, CD16, CD22, CD33, CD37, CD64, CD80, CD86, CD134, CD137, ICOS, CD154, and GITR can be used. The artificially designed transmembrane domain may be a polypeptide containing mainly hydrophobic residues such as leucine and valine. It is preferred that a triplet of phenylalanine, tryptophan and/or valine is found at each end of the artificial transmembrane domain. Optionally, a short oligopeptide linker or a polypeptide linker, for example a linker consisting of 2 to 10 amino acid sequence in length, can be placed between the transmembrane domain and the intracellular domain. Particularly, a linker sequence having a glycine-serine sequence can be used. In one embodiment of the present invention, the transmembrane domain can be a transmembrane domain having the sequence of amino acids 153 to 180 of CD28 (NCBI RefSeq: NP_006130.1). In another embodiment, a transmembrane domain having the sequence of amino acid numbers 162 to 183 of GITR (NCBI RefSeq: NP_004186.1) can be used.

(iii) Intracellular Domain

The CAR of the present invention comprises an intracellular domain. The intracellular domain is a molecule that can transmit a signal into a cell when the antigen-binding domain present in the same molecule binds to (interacts with) an antigen. It is known that signals generated through a T cell receptor (TCR) complex alone are insufficient for T cell activation, and secondary or costimulatory signals are also required. Natural T cell activation signals are transmitted by two distinct types of cytoplasmic signaling sequences: a sequence that initiates antigen-dependent primary activation via the TCR complex (activation domain) and a sequence that acts antigen-independently to provide a secondary or costimulatory signal (costimulatory domain). In a preferred aspect, the CAR of the present invention comprises the activation domain and/or costimulatory domain as the intracellular domain.

The activation domain may contain a signal transduction motif known as an immunoreceptor tyrosine-based activation motif (ITAM) [Nature, vol. 338, pp. 383-384 (1989)]. Examples of the activation domain that can be used in the present invention include ITAMs derived from CD3ζ, FcRγ, FcRβ, CD3γ, CD3δ, CD3ε, CD5, CD22, CD79a, CD79b, and CD66d. Specific examples of the activation domain include peptides having sequences of amino acid numbers 51-164 of CD3ζ (NCBI RefSeq: NP_932170.1), amino acid numbers 45-86 of FcεRIγ (NCBI RefSeq: NP_004097.1), amino acid numbers 201-244 of FceRIβ (NCBI RefSeq: NP_000130.1), amino acid numbers 139-182 of CD37 (NCBI RefSeq: NP_000064.1), amino acid numbers 128-171 of CD3δ (NCBI RefSeq: NP_000723.1), amino acid numbers 153-207 of CD3ε (NCBI RefSeq: NP_000724.1), amino acid numbers 402-495 of CD5 (NCBI RefSeq: NP_055022.2), amino acid numbers 707-847 of CD22 (NCBI RefSeq: NP_001762.2), amino acid numbers 166-226 of CD79a (NCBI RefSeq: NP_001774.1), amino acid numbers 182-229 of CD79b (NCBI RefSeq: NP_000617.1), and amino acid numbers 177-252 of CD66d (NCBI RefSeq: NP_001806.2), and their variants having the same functions as the peptides. The amino acid numbers based on the NCBI RefSeq ID and GenBank amino acid sequence information as used herein are numbers assigned to the full-length precursor (including a signal peptide sequence, etc.) of each protein.

The costimulatory domain comprises a sequence, for example, derived from CD2, CD4, CD5, CD8α, CD8β, CD28, CD134, CD137, ICOS, or CD154. Specific examples of the costimulatory domain include peptides having sequences of amino acid numbers 236-351 of CD2 (NCBI RefSeq: NP_001758.2), amino acid numbers 421-458 of CD4 (NCBI RefSeq: NP_000607.1), amino acid numbers 402-495 of CD5 (NCBI RefSeq: NP_055022.2), amino acid numbers 207-235 of CD8α (NCBI RefSeq: NP_001759.3), amino acid numbers 196-210 of CD80 (GenBank: AAA35664.1), amino acid numbers 181-220 of CD28 (NCBI RefSeq: NP_006130.1), amino acid numbers 214-255 of CD137 (4-1BB, NCBI RefSeq: NP_001552.2), amino acid numbers 241-277 of CD134 (OX40, NCBI RefSeq: NP_003318.1), and amino acid numbers 166-199 of ICOS (NCBI RefSeq: NP_036224.1), and their variants having the same functions as the peptides.

The present invention includes CARs that comprise one or more, for example two or three, activation domains or costimulatory domains as the intracellular domain. Particularly preferred examples of the CAR include a CAR comprising, as the intracellular domain, an activation domain of CD3ζ, and a CAR comprising an activation domain of CD3ζ and a costimulatory domain of CD28. The present invention also includes a CAR comprising tandem-connected multiple intracellular domains of the same type. In the CAR of the present invention, domains contained in the intracellular domain can be linked by inserting an oligopeptide linker or a polypeptide linker between the domains. Preferably, a linker consisting of 2 to 10 amino acids in length can be used. Particularly, a linker having a glycine-serine sequence can be used.

### (3) Nucleic Acid of the Present Invention

The present invention provides nucleic acids encoding the antibody or antigen-binding fragment thereof as described above in (1) and the CAR as described above in (2). The nucleic acid of the present invention is capable of expressing the antibody or antigen-binding fragment of the present invention, or the CAR of the present invention in a cell. Nucleic acids encoding the VL and VH regions of anti-BCMA antibody 1 of the present invention have, for example, the nucleic acid sequences of SEQ ID NOs: 49 and 50; nucleic acids encoding the VL and VH regions of anti-BCMA antibody 2 of the present invention have, for example, the nucleic acid sequences of SEQ ID NOs: 51 and 52; nucleic acids encoding the VL and VH regions of anti-BCMA antibody 3 of the present invention have, for example, the nucleic acid sequences of SEQ ID NOs: 53 and 54; nucleic acids encoding the VL and VH regions of anti-BCMA antibody 4 of the present invention have, for example, the nucleic acid sequences of SEQ ID NOs: 55 and 56; nucleic acids encoding the VL and VH regions of anti-BCMA antibody 5 of the present invention have, for example, the nucleic acid sequences of SEQ ID NOs: 57 and 58; and nucleic acids encoding the VL and VH regions of anti-BCMA antibody 6 of the present invention have, for example, the nucleic acid sequences of SEQ ID NOs: 59 and 60.

The type of nucleic acid of the present invention is not limited, and a single-stranded nucleic acid, a double-stranded nucleic acid, a DNA, or an RNA can be used. Examples of the DNA include a double-stranded DNA, a single-stranded DNA, and cDNA, and examples of RNA include mRNA.

The nucleic acid of the present invention can be linked to a nucleic acid having a promoter sequence so as to be expressed in cells. Examples of promoters include promoters that constitutively promote the expression of a gene or an operably linked construct, and promoters that induce the expression of a gene or an operably linked construct by the action of a drug or the like (e.g., tetracycline or doxorubicin). The nucleic acid of the present invention can also be linked to a nucleic acid comprising another regulatory element that cooperates with a promoter or a transcription initiation site, for example an enhancer sequence or a terminator sequence, to obtain efficient transcription of the nucleic acid.

In a certain embodiment, the nucleic acid of the present invention is codon-optimized for expression in a particular host cell.

### (4) Vector of the Present Invention

The present invention provides a vector containing the nucleic acid of the present invention as described above in (3). The vector of the present invention can deliver and express the antibody, antigen-binding fragment thereof, or CAR of the present invention into desired cells.

In an embodiment of the vector of the present invention, when two or more polypeptides are expressed, the two or more peptides may be transcribed and translated by separate promoters, or may be transcribed and translated by a single promoter using an internal ribosome entry site (IRES). Furthermore, the vector of the present invention may have a structure such that a nucleic acid encoding two or more polypeptides interconnected via a self-cleaving peptide is transcribed and translated by a single promoter.

The vector that can be used in the present invention contains a suitable control sequence operably linked to the nucleic acid of the present invention so that the nucleic acid of the present invention is expressed in a suitable host cell. Examples of the control sequence include a promoter for transcribing the nucleic acid of the present invention, an optional operator sequence for controlling transcription, a ribosome binding site sequence, an enhancer, a polyadenylation sequence, and a sequence for controlling the termination of transcription or translation. Examples of a promoter that controls transcription in mammalian cells include mammalian-derived promoters (PGK promoter, EF1-α promoter, β-globin promoter, etc.), virus-derived promoters (CMV promoter, SV40 promoter, MMLV-LTR promoter, HIV-LTR promoter, etc.), and artificially constructed promoters (CAG promoter, etc.). Furthermore, the vector may contain various sequences known to those skilled in the art, for example, a restriction enzyme cleavage site, a marker gene (selection gene) such as a drug resistance gene, a signal sequence, a leader sequence, and the like, as needed. These various sequences or sites may be appropriately selected and used by those skilled in the art depending on the type of polypeptide to be expressed, the host cell to be used, the culture medium, and other conditions.

The vector of the present invention may contain, in addition to the nucleic acid of the present invention, a gene that can serve as a marker for checking the introduction of the vector into a cell (e.g., a drug resistance gene, a gene encoding a reporter enzyme, or a gene encoding a fluorescent protein) or a therapeutic gene.

The vector that can be used in the present invention includes a vector that is integrated into the genome of a host cell, a vector that is not integrated into the genome of a host cell, and an episomal vector that exists in the cytoplasm and replicates autonomously. For example, a plasmid vector, a viral vector, a transposon vector, an artificial chromosome, etc. can be suitably used. Examples of the viral vector that can be used include retroviral vectors (including oncoretroviral vectors, lentiviral vectors, and pseudotype vectors), adenoviral vectors, adeno-associated virus (AAV) vectors, simian virus vectors, vaccinia virus vectors, Sendai virus vectors, Epstein-Barr virus (EBV) vectors, and HSV vectors. The viral vector preferably lacks the replication ability so that the virus cannot self-replicate in infected cells. Various viral vectors and methods for producing them are well known to those skilled in the art. Commercially available viral vectors may also be used in the present invention.

When used in combination with condensing agents such as liposomes and cationic lipids, non-viral vectors can also be used in the present invention. Furthermore, the nucleic acid of the present invention can be introduced into cells by calcium phosphate transfection, DEAE-dextran, electroporation, or particle bombardment.

### (5) Cells of the Present Invention

The present invention provides cells expressing the antibody or antigen-binding fragment thereof as described above in (1) and the CAR as described above in (2). The origin of the cell of the present invention is not particularly limited, and cells derived from mammals, for example, human cells, or cells derived from non-human mammals such as monkey, mouse, rat, pig, horse, and dog can be used. The cell of the present invention is preferably a human cell. The antibody of the present invention or an antigen-binding fragment thereof can be produced by culturing the cell of the present invention.

The type of cell of the present invention is not particularly limited, and any cells can be used. For example, cells harvested, isolated, or purified from body fluids, tissues, or organs, such as blood (peripheral blood, umbilical cord blood, etc.) or bone marrow, or cells obtained by differentiating the aforementioned cells or reprogramming the aforementioned cells to generate pluripotent stem cells (iPSCs) can be used (see, e.g., Themeli et al., 2013). Peripheral blood mononuclear cells (PBMCs), immune cells, umbilical cord blood mononuclear cells, fibroblasts, adipocyte precursors, hepatocytes, skin keratinocytes, mesenchymal stem cells, adipose stem cells, various cancer cell lines, or neural stem cells can be used. For example, NK cells or T cells, precursor cells of T cells (hematopoietic stem cells, lymphocyte precursor cells, etc.), or cell populations containing the aforementioned cells can be used. Examples of T cells include CD8-positive T cells, CD4-positive T cells, regulatory T cells, cytotoxic T cells, and tumor-infiltrating lymphocytes. A cell population containing T cells and precursor cells of T cells includes a PBMC. The above-mentioned cells may be collected from a living organism, obtained by expanding cells collected from a living organism, or established as a cell line. Furthermore, cells, for example immune cells, obtained by differentiation from pluripotent stem cells (ES cells, iPS cells, etc.) may also be used as the cell of the present invention. T cells can be obtained by producing iPS cells from a T cell clone having the ability to recognize and damage a specific type of cells, and then differentiating the iPS cells. The T cells thus obtained have the same specific cytotoxic activity as the starting T cell clone and are in a more undifferentiated state. Into such T cells, the nucleic acid encoding the chimeric receptor of the present invention is introduced to easily prepare cells that exhibit excellent therapeutic effects.

For the purpose of antibody production, the nucleic acid encoding the antibody of the present invention or the vector carrying the nucleic acid is introduced into cells suitable for producing recombinant proteins. Examples of such cells include Chinese hamster ovary (CHO) cell lines and immortalized cells originating from lymphoid tissues. For example, dihydrofolate reductase (DHFR)-deficient CHO cells are suitable host cells for antibody production.

As used herein, the term “immune cell” generally refers to any cells involved in immune function in a living body. Examples of immune cells include hematopoietic stem cells, neutrophils, basophils, macrophages, lymphocytes (B cells and T cells), monocytes, dendritic cells, natural killer (NK) cells, and plasma cells. Of these, T cells and NK cells are preferred for the present invention. As used herein, the term “immune cell” also includes “precursor cells of immune cells” having the ability to differentiate into the above-mentioned immune cells.

For desired transplantation of the cell of the present invention or a cell differentiated from the cell of the present invention into an organism, it is preferable that the nucleic acid is introduced into cells taken from the organism to be transplanted or an organism of the same species as the organism to be transplanted.

The cell of the present invention may express, in addition to the antibody or antigen-binding fragment thereof of the present invention or the CAR of the present invention, a polypeptide (enzyme, antibody, cytokine, receptor, etc.) or a nucleic acid (siRNA, etc.) useful for treating a disease.

As used herein, the term “target cell” refers to a cell desired to be reduced or eliminated in a patient, and examples thereof include tumor cells and cells infected with pathogens. Although not particularly limited, tumor cells are preferred, and hematopoietic tumor cells and solid tumor cells are more preferred.

The immune cell expressing the CAR of the present invention has the ability to recognize cells expressing BCMA and destroys the cells by the cytotoxic activity. The cytotoxic activity can be evaluated by a known method. For example, the cytotoxic activity of the cell of the present invention on a target cell labeled with a radioactive substance, a fluorescent substance, etc. can be evaluated by measuring the radioactivity or fluorescent intensity released from the target cells destroyed by the cell of the present invention. The cytotoxic activity of the cell of the present invention can be also detected by measuring the amounts of cytokines such as GM-CSF and IFN-γ that are specifically released from the cells of the present invention or target cells.

### (6) Cell Production Method of the Present Invention

The present invention provides a method for producing a cell, the method comprising a step of introducing the nucleic acid of the present invention as described above in (3) into cells. That is, the cell of the present invention as described above in (5) can be produced by the method for producing the cell of the present invention.

The above-mentioned step is usually carried out ex vivo. For example, the cell of the present invention can be produced by transforming cells in vitro using the nucleic acid as described above in (3) or the vector as described above in (4).

In the cell production method of the present invention, cells derived from mammals, for example, human-derived cells, or cells derived from non-human mammals such as monkey, mouse, rat, pig, cow, and dog can be used. The cells used in the method of the present invention are not particularly limited and any of the above-mentioned cells may be used. The above-mentioned cells may be collected from a living organism, obtained by expanding cells collected from a living organism, or established as a cell line. For desired transplantation of the produced cell or a cell differentiated from the produced cell into an organism, it is preferable that the nucleic acid or the vector is introduced into cells taken from the organism to be transplanted or an organism of the same species as the organism to be transplanted. Furthermore, the efficiency of nucleic acid introduction into cells can be improved by stimulating the cells before introduction of the nucleic acid or vector. A specific type of cells may be enriched. For example, when a PBMC is cultured in the presence of an anti-CD3 antibody, the proportion of T cells increases. Furthermore, when a PBMC is cultured in the presence of both an anti-CD3 antibody and a recombinant fibronectin fragment, RetroNectin, a cell population rich in CD8-positive T cells is obtained.

When used in combination with adjuvants such as liposomes, cationic lipids, or polyethyleneimine, non-viral vectors can also be used in the present invention. Furthermore, the nucleic acid of the present invention can be introduced into cells by calcium phosphate transfection, DEAE-dextran, electroporation, or particle bombardment.

Methods for producing various viral vectors and methods for introducing the vectors into cells are well known to those skilled in the art. For example, when a retroviral vector or a lentiviral vector is used, an appropriate packaging cell can be selected depending on an LTR sequence and a packaging signal sequence contained in the vector, and then used to prepare viral particles for gene transfer. Retroviral or lentiviral particles can also be produced using 293 cells or 293T cells which have high transfection efficiency. Many types of viral vectors produced on the basis of retroviruses and lentiviruses as well as packaging cells and packaging kits that can be used to package the vectors are commercially available from various companies.

The cell into which the nucleic acid of the present invention has been introduced can be cultured ex vivo to expand the cell number. For cell culture, a medium suitable for the cell may be used, and may be selected from known or commercially available media. For example, media that can be used to culture lymphocytes and other immune cells are known to those skilled in the art, and media with a variety of compositions are commercially available. These media include media containing a serum from human or an animal (e.g., fetal bovine serum; FBS or FCS), xeno-free media, and media containing no unknown component (defined media). A medium for the purpose may be selected. For cell culture, known culture vessels (plates, petri dishes, flasks, bags, culture tanks, etc.) can be used. The culture conditions may be the same as those for ordinary cell culture (for example, 32-37° C., 5% CO₂, etc.). Furthermore, at appropriate time intervals, dilution of the culture with a fresh medium, exchange of a medium, or exchange of a cell culture vessel may be carried out.

### (7) Pharmaceutical Composition of the Present Invention

The present invention provides a pharmaceutical composition comprising the cell of the present invention as described above in (5), for example, the immune cell expressing the CAR of the present invention, as an active ingredient. The pharmaceutical composition of the present invention may comprise a pharmaceutically acceptable excipient. Pharmaceutically acceptable excipients are well known to those skilled in the art. Examples of pharmaceutically acceptable excipients include phosphate buffered saline (e.g., 0.01 M phosphate, 0.138 M NaCl, 0.0027 M KCl, pH 7.4), aqueous solutions containing mineral acid salts such as hydrochlorides, hydrobromides, phosphates, sulfates, etc., saline, glycol solutions, ethanol solutions, and salts of organic acids such as acetates, propionates, malonates, benzoates, etc. Auxiliary substances, such as wetting or emulsifying agents, and pH buffering agents may also be used. Pharmaceutically acceptable excipients are also listed in Remington's Pharmaceutical Sciences (Mack Pub. Co., N.J. 1991). The pharmaceutical composition of the present invention can be in any known form suitable for parenteral administration, for example, injection or infusion. The pharmaceutical composition of the present invention may further contain formulation aids such as suspending agents, preservatives, stabilizers and/or dispersing agents, as well as preservatives for extending the shelf life during storage.

The present invention also provides pharmaceutical compositions that are different from cellular medicines. The antibody and antigen-binding fragment thereof of the present invention can be used to produce antibody-drug conjugates (ADCs) in combination with anticancer drugs or other drugs. The conjugates can specifically deliver the drug to cells, tissues, etc. expressing BCMA. The antibody or antigen-binding fragment may be bound to the drug by an appropriate means, taking into consideration the structure of the drug and other factors. Furthermore, the present invention can be used to produce bispecific antibodies. For example, a bispecific T cell-inducing antibody (BiTE) prepared by linking the antigen-binding fragment of the present invention to an antigen-binding fragment of an anti-CD3 antibody exerts an anti-tumor effect by inducing T cells to BCMA-expressing cells. Bispecific antibodies can be produced by recombinant DNA techniques using the nucleic acid of the present invention and a nucleic acid encoding another antibody or a fragment thereof.

### (8) Preventive or Therapeutic Method of Disease of the Present Invention

The present invention provides a method for preventing or treating a disease, the method comprising administering the pharmaceutical composition of the present invention as described above in (7) to a subject. The disease to be prevented or treated by administration of the pharmaceutical composition of the present invention, for example, the cell expressing the CAR of the present invention may be any disease sensitive to the cell contained in the composition as an active ingredient, and examples thereof include, but not limited to, diseases associated with cells expressing BMCA. Examples of the disease to be prevented or treated by the pharmaceutical composition of the present invention include B-cell-related diseases, such as multiple myeloma (MM), non-Hodgkin's lymphoma (NHL), B-cell proliferation of uncertain grade, lymphomatoid granulomatosis, post-transplant lymphoproliferative disorder, immunoregulatory disorders, rheumatoid arthritis, myasthenia gravis, idiopathic thrombocytopenic purpura, antiphospholipid syndrome, Chagas' disease, Graves' disease, Wegener's granulomatosis, polyarteritis nodosa, Sjogren's syndrome, pemphigus vulgaris, scleroderma, multiple sclerosis, antiphospholipid syndrome, ANCA-associated vasculitis, Goodpasture's disease, Kawasaki disease, autoimmune hemolytic anemia and rapidly progressive glomerulonephritis, heavy chain disease, primary or immune cell-associated amyloidosis, or monoclonal gammopathy of undetermined significance. MM includes overt multiple myeloma, smoldering multiple myeloma, plasma cell leukemia, non-secretory myeloma, IgD myeloma, osteosclerotic myeloma, solitary plasmacytoma of bone, and extramedullary plasmacytoma. NHL includes Burkitt's lymphoma, chronic lymphocytic leukemia/small lymphocytic lymphoma (CLL/SLL), diffuse large B-cell lymphoma, follicular lymphoma, immunoblastic large cell lymphoma, precursor B-lymphoblastic lymphoma, and mantle cell lymphoma.

The pharmaceutical composition may be administered intradermally, subcutaneously, intravenously, or by systemic administration or local administration to the affected area or tissue in the vicinity thereof, for example, subcutaneously. The dosage amount can be adjusted appropriately depending on the age and weight of the subject to which the pharmaceutical composition is administered, the administration method, etc. Further, the frequency of administration, and administration intervals for multiple administration are taken into consideration. The subject to which the pharmaceutical composition is administered is an animal, preferably a mammal, for example, a human, or a non-human mammal such as a monkey, mouse, rat, pig, horse, or dog. More preferably, the subject is a human.

According to the preventive or therapeutic method of disease of the present invention, it is possible to induce proliferation of the cell of the present invention in vivo and/or extend the survival period of the cell in vivo by administering a ligand that binds to the CAR expressed by the cell of the present invention to the subject to which the cell of the present invention has been administered. The ligand may be administered by any method. An appropriate method for administrating the ligand is selected taking into consideration the distribution of the cells of the present invention in vivo, etc. The dosage amount and administration schedule of the ligand may be appropriately determined taking into consideration the type and dosage amount of cells to be combined, the type of disease, and the like.

The present invention will be explained in more detail with reference to the following Examples to which the scope of the present invention is not limited.

### Example 1: Cloning of Anti-BCMA Antibody

Three BALB/c mice were immunized with Recombinant Human BCMA/TNFRSF17 Fc Chimera (R&D Systems, Cat. No. 193-BC) by an iliac lymph node method. Iliac lymph nodes were collected from the immunized mice. Lymphocytes were fused with SP2/0 myeloma cells, and antibody-producing hybridomas were cloned. Total RNA was extracted from six hybridoma clones producing anti-human BCMA antibodies using NucleoSpin RNA (Takara Bio Inc). A reverse transcription reaction (RT reaction) was carried out using 350 ng of the hybridoma-derived total RNA as a template, using SMARTer RACE 5′/3′ Kit (Takara Bio Inc.) to synthesize cDNA.

The isotypes of antibodies produced from the hybridomas were all determined to be IgG1 and κ. Thus, PCR of a DNA encoding a VL region was performed using a primer pair that was a mixture of a UPM sense primer (SEQ ID NO: 67: GATTACGCCAAGCTTCTCATTCCTGTTGAAGCTCTTGACAAT) corresponding to the 5′ end of 5′-RACE cDNA and an antisense primer (SEQ ID NO: 68: GATTACGCCAAGCTTCTCAATTTTCTTGTCCACCTTGGTGC) corresponding to the end of the L chain constant region of mIgG1, while PCR of a DNA encoding a VH region was performed using a primer pair that was a mixture of the UPM sense primer corresponding to the 5′ end of 5′-RACE cDNA and an antisense primer corresponding to the end of the H chain constant region CH1 of mIgG1. Specifically, PCR was performed using 2.5 μL of a solution after the RT reaction (cDNA solution) diluted 5.5-fold with a Tricine-EDTA buffer, to which 10× sense primer, 50 nM antisense primer, 2× SeqAmp Buffer, and SeqAmp DNA polymerase (Takara Bio Inc.) were added to make a total volume of 50 μL, by repeating 25 cycles of 94° C. for 30 seconds, 68° C. for 30 seconds, and 72° C. for 3 minutes in a TaKaRa PCR Thermal Cycler Dice (Takara Bio Inc.).

Next, PCR products were separated by agarose gel electrophoresis. Amplified variable region DNA fragments, i.e., a DNA encoding the LV region and a DNA encoding the HV region, were cloned. Specifically, after electrophoresis, a band corresponding to the amplified fragment was excised as a gel block, and the DNA was purified using NucleoSpin Gel and PCR Clean-up (Takara Bio Inc.), followed by an in-Fusion reaction with a cloning vector. Specifically, 50 ng of the cloning vector, 7.0 μL of the purified product from agarose gel, and 2.0 μL of In-Fusion HD Master Mix (Takara Bio Inc.) were mixed to make a total volume of 10 μL, and the mixture was reacted at 50° C. for 10 minutes in a TaKaRa PCR Thermal Cycler Dice. With 2.5 μL of the reaction mixture thus obtained, Escherichia coli (Stellar Competent Cells: Takara Bio Inc.) was transformed. Ten colonies obtained for each of the DNAs encoding the LV region and the HV region were selected and cultured. Plasmids were extracted from the culture using NucleoSpin Plasmid EasyPure and double-digested with restriction enzymes EcoRI and HindIII to confirm insertion of the amplified variable region DNA fragment.

The base sequences of the variable region DNA fragments were determined by sequencing the plasmids in which the insertion of the variable region DNA fragment was confirmed using 3.2 pmol of M13 reverse primer (SEQ ID NO: 69: CAGGAAACAGCTATGAC) and M13 forward primer (SEQ ID NO: 70: GTAAAACGACGGCCAGT).

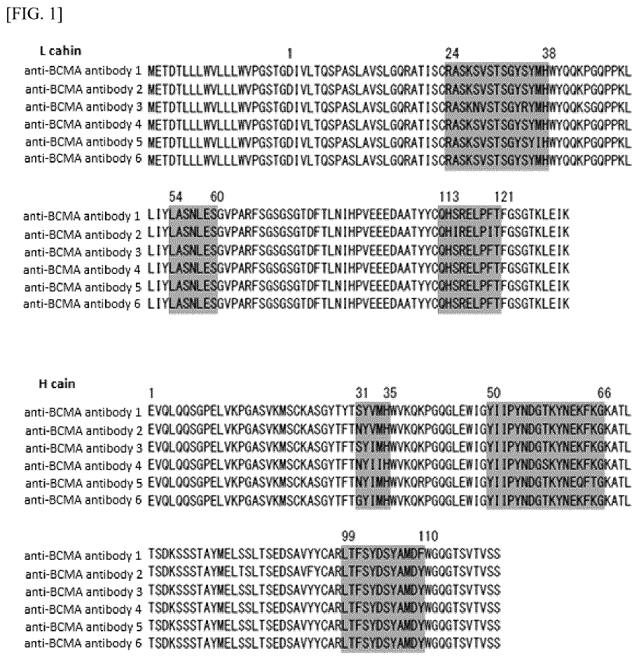

When the obtained base sequences were searched using BLAST, all clones showed homology to the base sequence of IgG light or heavy chain. Thus, it was confirmed that the cloned PCR products were DNA fragments containing nucleic acids encoding the variable regions of the light and heavy chains. An initiation codon ATG that was present upstream of the 5′ end of the constant region and that would result in the constant region being in frame was searched within the base sequence to deduce the amino acid sequence. The deduced amino acid sequences of the light and heavy chains of each clone were compared using ClustalW2 Multiple Sequence Alignment, and after adjusting to maximize homology, the complementarity-determining regions (CDRs) defined by Kabat were compared. As a result, CDR1 of the light chain was at amino acid positions 24-38, CDR2 was at amino acid positions 54-60, and CDR3 was at amino acid positions 113-121. On the other hand, CDR1 of the H chain was at amino acid positions 31-35, CDR2 was at amino acid positions 50-66, and CDR3 was at amino acid positions 99-110. Furthermore, from results of a series of analyses, it was estimated that the antibodies produced by the hybridoma clones were all different. The antibodies thus obtained are referred to as anti-BCMA antibodies 1 to 6. The CDR, VL, and VH sequences of anti-BCMA antibodies 1 to 6 are shown in Tables 2-1 to 2-5. A comparison of the amino acid sequences of anti-BCMA antibodies 1 to 6 is shown in FIG. 1.

TABLE 2-1

| SEQ ID | | |
|---|---|---|
| NO | Name | Sequence |
| 1 | Antibody 1 | RASKSVSTSGYSYMH |
| | VL CDR1 | |
| 2 | Antibody 1 | LASNLES |
| | VL CDR2 | |
| 3 | Antibody 1 | QHSRELPFT |
| | VL CDR3 | |
| 4 | Antibody 1 | SYVMH |
| | VH CDR1 | |
| 5 | Antibody 1 | YIIPYNDGTKYNEKFKG |
| | VH CDR2 | |
| 6 | Antibody 1 | LTFSYDSYAMDF |
| | VH CDR3 | |
| 7 | Antibody 2 | RASKSVSTSGYSYMH |
| | VL CDR1 | |
| 8 | Antibody 2 | LASNLES |
| | VL CDR2 | |
| 9 | Antibody 2 | QHIRELPIT |
| | VL CDR3 | |
| 10 | Antibody 2 | NYVMH |
| | VH CDR1 | |
| 11 | Antibody 2 | YIIPYNDGTKYNEKFKG |
| | VH CDR2 | |
| 12 | Antibody 2 | LTFSYDSYAMDY |
| | VH CDR3 | |
| 13 | Antibody 3 | RASKNVSTSGYRYMH |
| | VL CDR1 | |
| 14 | Antibody 3 | LASNLES |
| | VL CDR2 | |
| 15 | Antibody 3 | QHSRELPFT |
| | VL CDR3 | |
| 16 | Antibody 3 | SYIMHW |
| | VH CDR1 | |
| 17 | Antibody 3 | YIIPYNDGTKYNEKFKG |
| | VH CDR2 | |
| 18 | Antibody 3 | LTFSYDSYAMDY |
| | VH CDR3 | |

TABLE 2-2

| SEQ ID | | |
|---|---|---|
| NO | Name | Sequence |
| 19 | Antibody 4 | RASKSVSTSGYSYMH |
| | VL CDR1 | |
| 20 | Antibody 4 | LASNLES |
| | VL CDR2 | |
| 21 | Antibody 4 | QHSRELPFT |
| | VL CDR3 | |
| 22 | Antibody 4 | NYIIHW |
| | VH CDR1 | |
| 23 | Antibody 4 | YIIPYNDGSKYNEKFKG |
| | VH CDR2 | |
| 24 | Antibody 4 | LTFSYDSYAMDY |
| | VH CDR3 | |
| 25 | Antibody 5 | RASKSVSTSGYSYIH |
| | VL CDR1 | |
| 26 | Antibody 5 | LASNLES |
| | VL CDR2 | |
| 27 | Antibody 5 | QHSRELPFT |
| | VL CDR3 | |
| 28 | Antibody 5 | NYIMHW |
| | VH CDR1 | |
| 29 | Antibody 5 | YIIPYNDGTKYNEQFTG |
| | VH CDR2 | |
| 30 | Antibody 5 | LTFSYDSYAMDY |
| | VH CDR3 | |
| 31 | Antibody 6 | RASKSVSTSGYSYMH |
| | VL CDR1 | |
| 32 | Antibody 6 | LASNLES |
| | VL CDR2 | |
| 33 | Antibody 6 | QHSRELPFT |
| | VL CDR3 | |
| 34 | Antibody 6 | GYIMHW |
| | VH CDR1 | |
| 35 | Antibody 6 | YIIPYNDGTKYNEKFKG |
| | VH CDR2 | |
| 36 | Antibody 6 | LTFSYDSYAMDY |
| | VH CDR3 | |

TABLE 2-3

| SEQ ID | | |
|---|---|---|
| NO | Name | Sequence |
| 37 | Antibody 1 | DIVLTQSPASLAVSLGQRATISCRASKSVSTSGYSYMH |
| | VL chain amino | WYQQKPGQPPKLLIYLASNLESGVPARFSGSGSGTDF |
| | acid | TLNIHPVEEEDAATYYCQHSRELPFTFGSGTKLEIK |
| 38 | Antibody 1 | EVQLQQSGPELVKPGASVKMSCKASGYTYTSYVMH |
| | VH chain amino | WVKQKPGQGLEWIGYIIPYNDGTKYNEKFKGKATLTS |
| | acid | DKSSSTAYMELSSLTSEDSAVYYCARLTFSYDSYAMDF |
| | | WGQGTSVTVSS |
| 39 | Antibody 2 | DIVLTQSPASLAVSLGQRATISCRASKSVSTSGYSYMH |
| | VL chain amino | WYQQKPGQPPKLLIYLASNLESGVPARFSGSGSGTDF |
| | acid | TLNIHPVEEEDAATYYCQHIRELPITFGSGTKLEIK |
| 40 | Antibody 2 | EVQLQQSGPELVKPGASVKMSCKASGYTFTNYVMH |
| | VH chain amino | WVKQKPGQGLEWIGYIIPYNDGTKYNEKFKGKATLTS |
| | acid | DKSSSTAYMELTSLTSEDSAVFYCARLTFSYDSYAMDY |
| | | WGQGTSVTVSS |
| 41 | Antibody 3 | DIVLTQSPASLAVSLGQRATISCRASKNVSTSGYRYMH |
| | VL chain amino | WYQQKSGQPPKLLIYLASNLESGVPARFSGSGSGTDF |
| | acid | TLNIHPVEEEDAATYYCQHSRELPFTFGSGTKLEIK |
| 42 | Antibody 3 | EVQLQQSGPELVKPGASVKMSCKASGYTFTSYIMHW |
| | VH chain amino | VKQKPGQGLEWIGYIIPYNDGTKYNEKFKGKATLTSD |
| | acid | KSSSTAYMELSSLTSEDSAVYYCARLTFSYDSYAMDY |
| | | WGQGTSVTVSS |
| 43 | Antibody 4 | DIVLTQSPASLAVSLGQRATISCRASKSVSTSGYSYMH |
| | VL chain amino | WYQQKPGQPPRLLIYLASNLESGVPARFSGSGSGTDFT |
| | acid | LNIHPVEEEDAATYYCQHSRELPFTFGSGTKLEIK |
| 44 | Antibody 4 | EVQLQQSGPELVKPGASVKMSCKASGYTFTNYIIHWV |
| | VH chain amino | KQKPGQGLEWIGYIIPYNDGSKYNEKFKGKATLTSDK |
| | acid | SSSTAYMELSSLTSEDSAVYYCARLTFSYDSYAMDYW |
| | | GQGTSVTVSS |
| 45 | Antibody 5 | DIVLTQSPASLAVSLGQRATISCRASKSVSTSGYSYIHW |
| | VL chain amino | YQQKPGQPPKLLIYLASNLESGVPARFSGSGSGTDFTL |
| | acid | NIHPVEEEDAATYYCQHSRELPFTFGSGTKLEIK |
| 46 | Antibody 5 | EVQLQQSGPELVKPGASVKMSCKASGYTFTNYIMHW |
| | VH chain amino | VKQRPGQGLEWIGYIIPYNDGTKYNEQFTGKATLTSD |
| | acid | KSSSTAYMELSSLTSEDSAVYYCARLTFSYDSYAMDY |
| | | WGQGTSVTVSS |
| 47 | Antibody 6 | DIVLTQSPASLAVSLGQRATISCRASKSVSTSGYSYMH |
| | VL chain amino | WYQQKPGQPPKLLIYLASNLESGVPARFSGSGSGTDF |
| | acid | TLNIHPVEEEDAATYYCQHSRELPFTFGSGTKLEIK |
| 48 | Antibody 6 | EVQLQQSGPELVKPGASVKMSCKASGYTFTGYIMHW |
| | VH chain amino | VKQKPGQGLEWIGYIIPYNDGTKYNEKFKGKATLTSD |
| | acid | KSSSTAYMELSSLTSEDSAVYYCARLTFSYDSYAMDY |
| | | WGQGTSVTVSS |

TABLE 2-4

| SEQ ID | | |
|---|---|---|
| NO | Name | Sequence |
| 49 | Antibody 1 | GACATTGTGCTGACACAGTCTCCTGCTTCCTTAGCT |
| | VL chain | GTATCTCTGGGGCAGAGGGCCACCATCTCATGCAGG |
| | nucleic acid | GCCAGCAAAAGTGTCAGTACATCTGGCTATAGTTATA |
| | | TGCACTGGTACCAACAGAAACCAGGACAGCCACCC |
| | | AAACTCCTCATCTATCTTGCATCCAACCTAGAATCTG |
| | | GGGTCCCTGCCAGGTTCAGTGGCAGTGGGTCTGGG |
| | | ACAGACTTCACCCTCAACATCCATCCTGTGGAGGAG |
| | | GAGGATGCTGCAACCTATTACTGTCAGCACAGTAGG |
| | | GAGCTTCCATTCACGTTCGGCTCGGGGACAAAGTTG |
| | | GAAATAAAA |
| 50 | Antibody 1 | GAGGTCCAGCTGCAGCAGTCTGGACCTGAGCTGGT |
| | VH chain | AAAGCCTGGGGCTTCAGTGAAGATGTCCTGCAAGG |
| | nucleic acid | CTTCTGGATACACATACACTAGCTATGTTATGCACTG |
| | | GGTGAAGCAGAAGCCTGGGCAGGGCCTTGAGTGGA |
| | | TTGGATATATTATTCCTTACAATGATGGTACTAAGTAC |
| | | AATGAGAAGTTCAAGGGCAAGGCCACACTGACTTC |
| | | AGACAAATCCTCCAGCACAGCCTACATGGAGCTCAG |
| | | CAGCCTGACCTCTGAGGACTCTGCGGTCTATTACTG |
| | | TGCAAGATTGACCTTTTCTTACGACTCCTATGCTATG |
| | | GACTTCTGGGGTCAAGGAACCTCAGTCACCGTCTCC |
| | | TCA |
| 51 | Antibody 2 | GACATTGTGCTGACACAGTCTCCTGCTTCCTTAGCT |
| | VL chain | GTATCTCTGGGGCAGAGGGCCACCATCTCATGCAGG |
| | nucleic acid | GCCAGCAAAAGTGTCAGTACATCTGGTTATAGTTATA |
| | | TGCACTGGTACCAACAGAAACCAGGACAGCCACCC |
| | | AAACTCCTCATCTATCTTGCATCCAACCTAGAATCTG |
| | | GGGTCCCTGCCAGGTTCAGTGGCAGTGGGTCTGGG |
| | | ACAGACTTCACCCTCAACATCCATCCTGTGGAGGAG |
| | | GAGGATGCTGCAACGTATTACTGTCAGCACATTAGG |
| | | GAGCTTCCAATCACGTTCGGCTCGGGGACAAAGTT |
| | | GGAAATAAAA |
| 52 | Antibody 2 | GAGGTCCAGCTGCAGCAGTCTGGACCTGAGCTGGT |
| | VH chain | AAAGCCTGGGGCTTCAGTGAAGATGTCCTGCAAGG |
| | nucleic acid | CTTCTGGATACACATTCACTAACTATGTTATGCACTG |
| | | GGTGAAGCAGAAGCCTGGGCAGGGCCTTGAGTGGA |
| | | TTGGATATATTATTCCTTATAATGATGGTACTAAATAC |
| | | AATGAGAAGTTCAAAGGCAAGGCCACACTGACTTC |
| | | AGACAAATCCTCCAGCACAGCCTACATGGAGCTCAC |
| | | CAGCCTGACCTCTGAGGACTCTGCGGTCTTTTACTG |
| | | TGCAAGATTGACCTTTTCTTACGACTCCTATGCTATG |
| | | GACTACTGGGGTCAAGGAACCTCAGTCACCGTCTCC |
| | | TCA |
| 53 | Antibody 3 | GACATTGTGCTGACACAGTCTCCTGCTTCCTTAGCT |
| | VL chain | GTATCTCTGGGGCAGAGGGCCACCATCTCATGCAGG |
| | nucleic acid | GCCAGCAAAAATGTCAGTACATCTGGCTATAGGTAC |
| | | ATGCACTGGTACCAACAGAAATCAGGACAGCCACC |
| | | CAAACTCCTCATCTATCTTGCATCCAACCTAGAATCT |
| | | GGGGTCCCTGCCAGGTTCAGTGGCAGTGGGTCTGG |
| | | GACAGACTTCACCCTCAACATCCATCCTGTGGAGGA |
| | | GGAGGATGCTGCAACCTATTACTGTCAGCACAGTAG |
| | | GGAGCTTCCATTCACGTTCGGCTCGGGGACAAAGTT |
| | | GGAAATAAAA |
| 54 | Antibody 3 | GAGGTCCAGCTGCAGCAGTCTGGACCTGAGCTGGT |
| | VH chain | AAAGCCTGGGGCTTCAGTGAAGATGTCCTGCAAGG |
| | nucleic acid | CTTCTGGATACACATTCACTAGCTATATTATGCACTG |
| | | GGTGAAGCAGAAGCCTGGGCAGGGCCTTGAGTGGA |
| | | TTGGATATATTATTCCTTACAATGATGGTACTAAGTAC |
| | | AATGAGAAGTTCAAAGGCAAGGCCACACTGACTTC |
| | | AGACAAATCCTCCAGCACAGCCTACATGGAGCTCAG |
| | | CAGCCTGACCTCTGAGGACTCTGCGGTCTATTACTG |
| | | TGCAAGATTGACCTTTTCTTACGACTCCTATGCTATG |
| | | GACTACTGGGGTCAAGGAACCTCAGTCACCGTCTCC |
| | | TCA |

TABLE 2-5

| SEQ ID | | |
|---|---|---|
| NO | Name | Sequence |
| 55 | Antibody 4 | GACATTGTGCTGACACAGTCTCCTGCTTCCTTAGCT |
| | VL chain | GTATCTCTGGGGCAGAGGGCCACCATCTCATGCAGG |
| | nucleic acid | GCCAGCAAAAGTGTCAGTACATCTGGCTATAGTTATA |
| | | TGCACTGGTACCAACAGAAACCAGGACAGCCACCC |
| | | AGACTCCTCATCTATCTTGCATCCAACCTAGAATCTG |
| | | GGGTCCCTGCCAGGTTCAGTGGCAGTGGGTCTGGG |
| | | ACAGACTTCACCCTCAACATCCATCCTGTGGAGGAG |
| | | GAGGATGCTGCAACCTATTACTGTCAGCACAGTAGG |
| | | GAGCTTCCATTCACGTTCGGCTCGGGGACAAAGTTG |
| | | GAAATAAAA |
| 56 | Antibody 4 | GAGGTCCAGCTGCAGCAGTCTGGACCTGAGCTGGT |
| | VH chain | AAAGCCTGGGGCTTCAGTGAAGATGTCCTGCAAGG |
| | nucleic acid | CTTCTGGATACACATTCACTAACTATATTATACACTGG |
| | | GTGAAGCAGAAGCCTGGGCAGGGCCTTGAGTGGAT |
| | | TGGATATATTATTCCTTACAATGATGGTTCTAAGTACA |
| | | ATGAGAAGTTCAAAGGCAAGGCCACACTGACTTCA |
| | | GACAAATCCTCCAGCACAGCCTACATGGAGCTCAGC |
| | | AGCCTGACCTCTGAGGACTCTGCGGTCTATTACTGT |
| | | GCAAGATTGACCTTTTCTTACGACTCCTATGCTATGG |
| | | ACTACTGGGGTCAAGGAACCTCAGTCACCGTCTCCT |
| | | CA |
| 57 | Antibody 5 | GACATTGTGCTGACACAGTCTCCTGCTTCCTTAGCT |
| | VL chain | GTATCTCTGGGGCAGAGGGCCACCATCTCATGCAGG |
| | nucleic acid | GCCAGCAAAAGTGTCAGTACATCTGGCTATAGTTATA |
| | | TACACTGGTACCAACAGAAACCAGGACAGCCACCC |
| | | AAACTCCTCATCTATCTTGCATCCAACCTAGAATCTG |
| | | GGGTCCCTGCCAGGTTCAGTGGCAGTGGGTCTGGG |
| | | ACAGACTTCACCCTCAACATCCATCCTGTGGAGGAG |
| | | GAGGATGCTGCAACCTATTACTGTCAGCACAGTAGG |
| | | GAGCTTCCATTCACGTTCGGCTCGGGGACAAAGTTG |
| | | GAAATAAAA |
| 58 | Antibody 5 | GAGGTCCAGCTGCAGCAGTCTGGACCTGAGCTGGT |
| | VH chain | AAAGCCTGGGGCTTCAGTGAAGATGTCCTGCAAGG |
| | nucleic acid | CTTCTGGATACACATTCACTAACTATATTATGCACTG |
| | | GGTGAAGCAGAGGCCTGGGCAGGGCCTTGAGTGGA |
| | | TTGGATATATTATTCCTTACAATGATGGTACTAAGTAC |
| | | AATGAGCAGTTCACAGGCAAGGCCACACTGACTTC |
| | | AGACAAATCCTCCAGCACAGCCTACATGGAGCTCAG |
| | | CAGCCTGACCTCTGAGGACTCTGCGGTCTATTACTG |
| | | TGCAAGATTGACCTTTTCTTACGACTCCTATGCTATG |
| | | GACTACTGGGGTCAAGGAACCTCAGTCACCGTCTCC |
| | | TCA |
| 59 | Antibody 6 | GACATTGTGCTGACACAGTCTCCTGCTTCCTTAGCT |
| | VL chain | GTATCTCTGGGGCAGAGGGCCACCATCTCATGCAGG |
| | nucleic acid | GCCAGCAAAAGTGTCAGTACATCTGGCTATAGTTATA |
| | | TGCACTGGTACCAACAGAAACCAGGACAGCCACCC |
| | | AAACTCCTCATCTATCTTGCATCCAACCTAGAATCTG |
| | | GGGTCCCTGCCAGGTTCAGTGGCAGTGGGTCTGGG |
| | | ACAGACTTCACCCTCAACATCCATCCTGTGGAGGAG |
| | | GAGGATGCTGCAACCTATTACTGTCAGCACAGTAGG |
| | | GAGCTTCCATTCACGTTCGGCTCGGGGACAAAGTTG |
| | | GAAATAAAA |
| 60 | Antibody 6 | GAGGTCCAGCTGCAGCAGTCTGGACCTGAGCTGGT |
| | VH chain | AAAACCTGGGGCTTCTGTGAAGATGTCCTGCAAGG |
| | nucleic acid | CTTCTGGATACACATTCACTGGCTATATTATGCACTG |
| | | GGTGAAGCAGAAGCCTGGGCAGGGCCTTGAGTGGA |
| | | TTGGATATATTATTCCTTACAATGATGGTACTAAGTAC |
| | | AATGAGAAGTTCAAAGGCAAGGCCACACTGACTTC |
| | | AGACAAATCCTCCAGCACAGCCTACATGGAGCTCAG |
| | | CAGCCTGACCTCTGAGGACTCTGCGGTCTATTACTG |
| | | TGCAAGATTGACCTTTTCTTACGACTCCTATGCTATG |
| | | GACTACTGGGGTCAAGGAACCTCAGTCACCGTCTCC |
| | | TCA |

### Example 2: Anti-BCMA CAR

### (1) Construction of CAR Expression Vector Plasmid

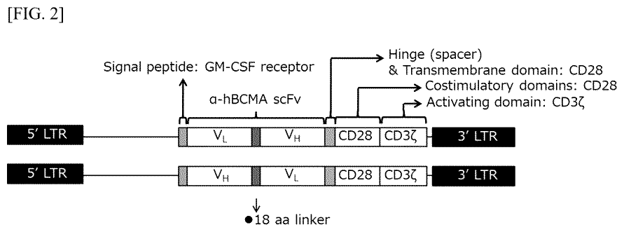

CARs containing antigen-binding fragments of each anti-BCMA antibody obtained in Example 1 were prepared. A nucleic acid encoding a CAR was prepared, the CAR containing, from the N-terminus, the signal peptide sequence of a GM-CSF receptor, the VL region of the anti-BCMA antibody, a linker sequence (18 amino acids, SEQ ID NO: 64, also referred to as “18aa linker”), the VH region of the anti-BCMA antibody, the extracellular region, transmembrane region and intracellular region of CD28, and the intracellular region of CD3ζ. This CAR was named BCMA-CAR. A nucleic acid encoding a CAR in which the order of the VH and VL regions was reversed was also prepared. For distinguishing the order of the VH and VL regions, the CARs are referred to as an LH construct or HL construct of BCMA-CAR. Furthermore, for distinguishing linker lengths, for example, the CAR containing an 18-amino acid linker is referred to as an L18H construct or an H18L construct of BCMA-CAR. The structures of these CARs are shown in FIG. 2. The nucleic acid was inserted into a pMEI-5 DNA vector (Takara Bio Inc.) to prepare retroviral plasmid pMEI-5/BCMA-CAR.

### (2) Preparation of Retroviral Vector Solution

Unless otherwise specified, the 293T cell line and the PG13 cell line (ATCC CRL-10686) were cultured in a DMEM/F-12 medium supplemented with 10% fetal bovine serum (FBS) and 100 U/mL penicillin-streptomycin.

Retroviral vector solutions were prepared using the various retroviral plasmids prepared in Example 2-(1), according to the procedures described below. First, a transient retroviral vector supernatant was prepared with 293T cells. Retroviral packaging plasmids (Gag-pol expression plasmid pGP, VSV-G expression plasmid pVSV-G (Uchibori R, et al. Mol Ther Oncolytics. 218; 12:16-25)) and the retroviral plasmid pMEI-5/BCMA-CAR were introduced into 293 T cells by the calcium phosphate method, and the cells were incubated for 7 hours at 37° C. and 5% CO₂. The entire medium was replaced with a DMEM/F-12 medium supplemented with sodium butyrate (STEMCELL Technologies Inc.) at a final concentration of 5 mM, and the cells were cultured for 16 hours at 37° C. and 5% CO₂. The entire medium was replaced with a DMEM/F-12 medium, and the cells were incubated for 24 hours at 32° C. and 5% CO₂. After the incubation, the cells were centrifuged at 2,000×g for 10 minutes to collect a viral vector supernatant. The supernatant was then filtered through a 0.45 m filter to remove cell debris. This virus supernatant was mixed with Retro-X Concentrator (Takara Bio Inc.), and then left to stand overnight at 4° C. The mixture was centrifuged at 2,000×g for 10 minutes, and a pellet was resuspended in 1 mL of a DMEM/F-12 medium to obtain a concentrated virus solution.

Next, retroviral vectors were produced using the PG13 cell line as a packaging cell line. Specifically, PG13 cells seeded onto a 6-well plate were exposed to the purified virus solution in the presence of 8 g/mL polybrene, and left to stand in an incubator set at 37° C. and 5% CO₂ for 7 hours. After 1 mL of a DMEM/F-12 medium was added, the plate was left to stand in the incubator overnight. The next day, the entire medium was replaced with a fresh medium and the cells were left to stand in the incubator set at 37° C. and 5% CO₂ for 6 hours. Then, the transduced cells were detached and the entire volume was subcultured into a T-75 flask. When the cells reached subconfluence, they were cultured for 7 to 10 days in an incubator set at 37° C. and 5% CO₂ to isolate single cell clones by limiting dilution. The cells were detached from the wells by trypsinization and seeded onto a 12-well plate, and cultured for 3 to 4 days in an incubator set at 37° C. and 5% CO₂. The cells were detached from the wells by trypsinization, and three-quarters of the cells were resuspended in a cryopreservation solution (CELLBANKER; Nippon Zenyaku Kogyo Co., Ltd.) and frozen and stored in a −80° C. freezer. The remaining ¼ of the cells were seeded onto a 12-well plate and cultured for 3 to 4 days in an incubator set at 37° C. and 5% CO₂. When the cells reached confluence, the entire medium was replaced with a DMEM/F-12 medium supplemented with sodium butyrate at a final concentration of 5 mM. The cultured cells were left to stand in an incubator set at 32° C. and 5% CO₂ for 48 hours. The cultured cells were centrifuged at 2,000×g for 10 minutes. A supernatant was collected and filtered through a 0.45 m filter to remove cell debris to obtain a retroviral vector solution. A portion of the retrovirus vector solution was taken and subjected to measurement of titer (Retrovirus Titer Set (for Real Time PCR): Takara Bio Inc.). The remainder of the retrovirus vector solution was dispensed into cryogenic tubes and stored frozen in a −80° C. freezer until use. The viral supernatant titer of each clone (Retrovirus Titer Set (for Real-Time PCR)) was measured and selected to establish a clone strain stably producing retroviral vectors. For clones stably producing retroviral vectors with a viral titer of 4.0×10⁹ copies/mL or more in the supernatant, thawed cryopreserved cells were seeded into a T-75 flask and then expanded to a T-225 flask. When the cells reached confluence, the medium was replaced with a DMEM/F-12 medium supplemented with sodium butyrate at a final concentration of 5 mM. The retroviral vector solution was collected in the same manner as described above, dispensed into cryogenic tubes, and frozen and stored in a −80° C. freezer until use.

### (3) Preparation of Jurkat Cell for Anti-BCMA-CAR Functional Assay

Jurkat cell line (ECACC EC88042803) and its transduced cells were cultured in a RPMI1640 medium supplemented with 10% fetal bovine serum (FBS) and 100 U/mL penicillin-streptomycin, unless otherwise specified.

A solution of a self-inactivating retroviral vector carrying an expression cassette having a switch function for inducing the expression of a luciferase gene in response to an activation signal from CAR was prepared by the procedures described below. The expression cassette can be used to assay the function of BCMA-CAR. First, a transient retroviral vector supernatant was prepared with 293T cells. Retroviral packaging plasmids (Gag-pol expression plasmid pGP, VSV-G expression plasmid pVSV-G) and retroviral plasmid pRetro-X Q/BR6N-ELuc were introduced into 293T cells by the calcium phosphate method, and the cells were incubated for 7 hours at 37° C. and 5% CO₂ (Uchibori R, et al. Mol Ther Oncolytics. 2018; 12:16-25). The entire medium was replaced with a DMEM/F-12 medium supplemented with sodium butyrate (STEMCELL Technologies Inc.) at a final concentration of 5 mM, and the cells were incubated for 16 hours at 37° C. and 5% CO₂. Then, the entire medium was replaced with a DMEM/F-12 medium, and the cells were incubated for 24 hours at 32° C. and 5% CO₂. After the incubation, the cells were centrifuged at 2,000×g for 10 minutes to collect a viral vector supernatant. The supernatant was then filtered through a 0.45 m filter to remove cell debris.

### (4) Functional Assay of Anti-BCMA-CAR

Jurkat cells were transduced using the RetroNectin-conjugated virus infection method (J. Biochem. 2001 September;130(3):331-4). A 20 μg/mL RetroNectin solution (Takara Bio Inc.) diluted with PBS was added to an untreated 24-well plate at 0.5 mL/well, and the plate was left at 4° C. The RetroNectin solution was removed, and 0.5 mL of a blocking solution (phosphate-buffered saline (PBS) supplemented with 2% bovine serum albumin (BSA)) was added to the plate. The plate was left to stand at room temperature for 30 minutes for blocking. The blocking solution was removed, 0.5 mL/well of PBS was added to wash the plate once, and the PBS was then removed. The BR6N-ELuc retroviral vector solution prepared in Example 2-(3) (diluted with PBS to 4.0×10⁹ copies/mL) was added to each well of a RetroNectin-coated plate at 0.5 mL/well. The plate containing the retrovirus was placed in a centrifuge kept at 32° C. and centrifuged at 32° C. and 1,960 xg for 2 hours to promote adsorption of the virus particles onto RetroNectin. The virus dilution was removed from the plate, and the plate was washed with 1 mL of PBS containing 1% BSA. Jurkat cells (diluted with a RPMI 1640 medium to 4.0×10⁵ cells/mL) were added to the virus-adsorbed plate at 1 mL/well, and the plate was centrifuged at 32° C. and 1,960×g for 10 minutes. The plate was left overnight in an incubator set at 37° C. and 5% CO₂ to allow for transduction. The transduced Jurkat cells were collected the next day, suspended in 7.5 mL of a RPMI 1640 medium, and placed in an incubator set at 37° C. and 5% CO₂ (Day 1). When the cells reached subconfluence, the cells were cultured for 7 to 10 days in an incubator set at 37° C. and 5% CO₂ to isolate single cell clones by limiting dilution. The cells were collected from the well, seeded onto a 12-well plate, and cultured for 3 to 4 days in an incubator set at 37° C. and 5% CO₂. The cells were collected from the well, seeded into a T-25 flask, and cultured for 3 to 4 days in an incubator set at 37° C. and 5% CO₂. Of the cells 1.0×10⁵ cells/well were seeded into a 96-well white microplate for luminescence signal detection. The remaining cells were seeded into a T-75 flask and continued to be cultured. TPA (12-O-tetradecanoylphorbol 13-acetate) and ionomycin were added to the cells seeded in the 96-well white microplate, and a luciferase assay was performed 24 hours later to measure luminescence intensity. Cells showing a low luminescence intensity in the non-stimulation group and an excellent luminescence intensity ratio between the non-stimulation group and the stimulation group were used as transduced Jurkat cells (Jurkat/iELuc cells) to be used in a functional assay of BCMA-CAR. The Jurkat/iELuc cells were expanded in a T-225 flask. When the cells reached confluence, the cells were suspended in a cryopreservation solution (CELLBANKER; Nippon Zenyaku Kogyo Co., Ltd.), dispensed into cryogenic tubes, and frozen and stored in a −80° C. freezer until use.

(5) Transduction of Anti-BCMA-CAR Construct into Jurkat/iELuc Cell

### (Functional Assay of Anti-BCMA-CAR)

The RetroNectin-conjugated virus infection method was used for transduction into the Jurkat/iELuc cells. The Jurkat/iELuc cells (diluted to 4.0×10⁵ cells/mL with a RPMI1640 medium) were added at 1 mL/well to a RetroNectin-coated 24-well plate onto which the BCMA-CAR retrovirus had been adsorbed using the same procedure as in Example 2-(4), and the plate was centrifuged at 32° C. and 1,960×g for 10 minutes. The plate was left overnight in an incubator set at 37° C. and 5% CO₂ to allow for transduction. The transduced Jurkat/iELuc cells were collected the next day, suspended in 7.5 mL of a RPMI 1640 medium, and placed in an incubator set at 37° C. and 5% CO₂ (Day 1).

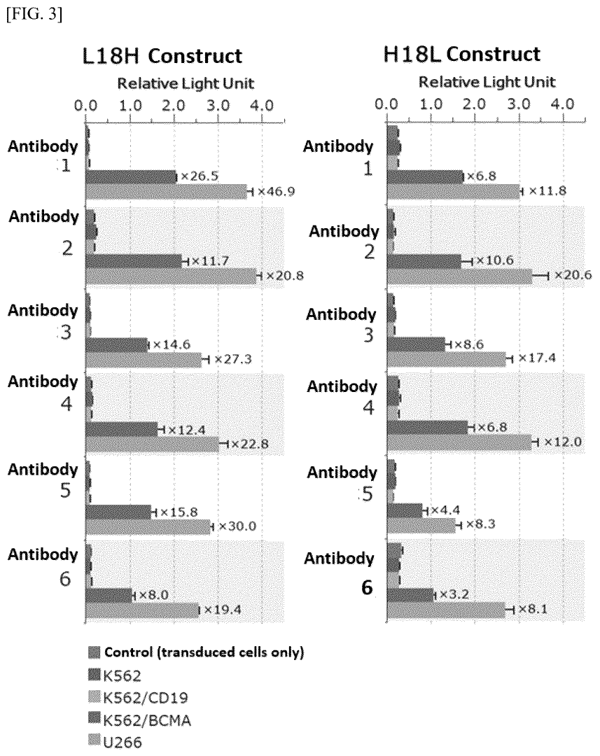

The transduced cells (GMCs) thus produced were collected on Day 7, and mixed with chronic myeloid leukemia cell line K562 cells, BCMA-expressing K562 cells (K562-BCMA), or BCMA-expressing human myeloma cell line U266 cells at an effector:target ratio of 1:1 in a 96-well white microplate for luminescence signal detection, and co-cultured for 18 hours in an incubator set at 37° C. and 5% CO₂. Bright-Glo Luciferase Assay reagent (Promega) was added, and the luminescence intensity of the transduced cells was measured. Results are shown in FIG. 3. As shown in FIG. 3, a BCMA-specific increase in luminescence intensity was observed in the Jurkat/iELuc cells transduced with BCMA-CAR. Particularly, the LH construct derived from anti-BCMA antibody 1 had the highest reactivity, with low luminescence intensity of the control (transduced cells only) and the luminescence intensity ratio of the U266 co-culture group to the control group being 46.9 times.

### (6) Comparison of Linker Length

Retroviral plasmids were constructed for the LH construct derived from anti-BCMA antibody 1 using the same procedures as in Example 2-(1) except that the linker amino acid length was replaced with 15 residues (L15H, SEQ ID NO: 62) or 20 residues (L20H, SEQ ID NO: 63), and their retroviral vector solutions were prepared. The Jurkat/iELuc cells were transduced with the retroviral vectors using the same procedures as in Example 2-(2) to (5), and then mixed with K562 cells, K562-BCMA cells, or U266 cells at an effector:target ratio of 1:1 in a 96-well white microplate for luminescence signal detection, and co-cultured for 18 hours in an incubator set at 37° C. and 5% CO₂. As a control, non-transduced cells (Jurkat cells and Jurkat/iELuc cells) were also co-cultured in the same manner. Bright-Glo Luciferase Assay reagent (Promega) was added, and the luminescence intensity of the transduced cells was measured. Results are shown in FIG. 4. As shown in FIG. 4, a BCMA-specific increase in luminescence intensity was observed in the Jurkat/iELuc cells transduced with BCMA-CAR. Particularly, the L20H construct had the highest reactivity, with low luminescence intensity of the control (transduced cells only) and the luminescence intensity ratio of the U266 co-culture group to the control group being 62.8 times. The CAR having the LH construct derived from antibody 1 and having the linker length of 20 amino acids is designated BCMA-CALR (L20H), and its amino acid sequence is shown in SEQ ID NO: 65. The nucleotide sequence of a nucleic acid encoding BCMA-CALR (L20H) is shown in SEQ ID NO: 66 (Table 3).

TABLE 3

| SEQ ID | | |
|---|---|---|
| NO | Name | Sequence |
| 65 | BCMA- | MLLLVTSLLLCELPHPAFLLIPDIVLTQSPASLAVSLGQRATIS |
| | CAR | CRASKSVSTSGYSYMHWYQQKPGQPPRLLIYLASNLESGV |
| | (L20H) | PARFSGSGSGTDFTLNIHPVEEEDAATYYCQHSRELPFTFGS |
| | amino | GTKLEIKGGGGSGGGGSGGGGSGGGGSEVQLQQSGPELVK |
| | acid | PGASVKMSCKASGYTFTNYIIHWVKQKPGQGLEWIGYIIPY |
| | | NDGSKYNEKFKGKATLTSDKSSSTAYMELSSLTSEDSAVYY |
| | | CARLTFSYDSYAMDYWGQGTSVTVSSATGAAAIEVMYPPP |
| | | YLDNEKSNGTIIHVKGKHLCPSPLFPGPSKPFWVLVVVGGV |
| | | LACYSLLVTVAFIIFWVRSKRSRLLHSDYMNMTPRRPGPTR |
| | | KHYQPYAPPRDFAAYRSRVKFSRSADAPAYQQGQNQLYNE |
| | | LNLGRREEYDVLDKRRGRDPEMGGKPRRKNPQEGLYNEL |
| | | QKDKMAEAYSEIGMKGERRRGKGHDGLYQGLSTATKDTY |
| | | DALHMQALPPR |
| 66 | BCMA- | ATGCTGCTCCTGGTGACCAGCTTGCTGCTGTGCGAGCTTC |
| | CAR | CTCACCCAGCCTTCCTCCTGATCCCCGACATTGTGCTGAC |
| | (L20H) | ACAGTCTCCTGCTTCCTTAGCTGTATCTCTGGGGCAGAGG |
| | nucleic | GCCACCATCTCATGCAGGGCCAGCAAAAGTGTCAGTACA |
| | acid | TCTGGCTATAGTTATATGCACTGGTACCAACAGAAACCAG |
| | | GACAGCCACCCAGACTCCTCATCTATCTTGCATCCAACCT |
| | | AGAATCTGGGGTCCCTGCCAGGTTCAGTGGCAGTGGGTC |
| | | TGGGACAGACTTCACCCTCAACATCCATCCTGTGGAGGA |
| | | GGAGGATGCTGCAACCTATTACTGTCAGCACAGTAGGGA |
| | | GCTTCCATTCACGTTCGGCTCGGGGACAAAGTTGGAAAT |
| | | AAAAGGGGGAGGAGGATCTGGTGGAGGAGGGAGTGGCG |
| | | GTGGCGGATCTGGAGGTGGAGGAAGCGAGGTCCAGCTG |
| | | CAGCAGTCTGGACCTGAGCTGGTAAAGCCTGGGGCTTCA |
| | | GTGAAGATGTCCTGCAAGGCTTCTGGATACACATTCACTA |
| | | ACTATATTATACACTGGGTGAAGCAGAAGCCTGGGCAGG |
| | | GCCTTGAGTGGATTGGATATATTATTCCTTACAATGATGGT |
| | | TCTAAGTACAATGAGAAGTTCAAAGGCAAGGCCACACTG |
| | | ACTTCAGACAAATCCTCCAGCACAGCCTACATGGAGCTC |
| | | AGCAGCCTGACCTCTGAGGACTCTGCGGTCTATTACTGTG |
| | | CAAGATTGACCTTTTCTTACGACTCCTATGCTATGGACTAC |
| | | TGGGGTCAAGGAACCTCAGTCACCGTCTCCTCAGCTaccgg |
| | | tgcggccgcaATTGAAGTTATGTATCCTCCTCCTTACCTAGACA |
| | | ATGAGAAGAGCAATGGAACCATTATCCATGTGAAAGGGA |
| | | AACACCTTTGTCCAAGTCCCCTATTTCCCGGACCTTCTAA |
| | | GCCCTTTTGGGTGCTGGTGGTGGTTGGGGGAGTCCTGGC |
| | | TTGCTATAGCTTGCTAGTAACAGTGGCCTTTATTATTTTCT |
| | | GGGTGAGGAGTAAGAGGAGCAGGCTCCTGCACAGTGAC |
| | | TACATGAACATGACTCCCCGCCGCCCCGGGCCCACCCGC |
| | | AAGCATTACCAGCCCTATGCCCCACCACGCGACTTCGCA |
| | | GCCTATCGCTCCAGAGTGAAGTTCAGCAGGAGCGCAGAC |
| | | GCCCCCGCGTACCAGCAGGGCCAGAACCAGCTCTATAAC |
| | | GAGCTCAATCTAGGACGAAGAGAGGAGTACGATGTTTTG |
| | | GACAAGAGACGTGGCCGGGACCCTGAGATGGGGGGAAA |
| | | GCCGAGAAGGAAGAACCCTCAGGAAGGCCTGTACAATG |
| | | AACTGCAGAAAGATAAGATGGCGGAGGCCTACAGTGAG |
| | | ATTGGGATGAAAGGCGAGCGCCGGAGGGGCAAGGGGCA |
| | | CGATGGCCTTTACCAGGGTCTCAGTACAGCCACCAAGGA |
| | | CACCTACGACGCCCTTCACATGCAGGCCCTGCCCCCTCG |
| | | CTAA |

### Example 3: Evaluation of Anti-BCMA-CAR

(1) Transduction of Anti-BCMA-CAR Construct into T Cell

Peripheral blood was collected from volunteers who gave informed consent into BD Vacutainer CPT cell preparation tubes for the separation of mononuclear cells, and separated into peripheral blood mononuclear cells (PBMCs) and plasma by centrifugation at 25° C. and 1,500×g for 15 minutes. The PBMCs were washed twice with CELLOTION (Nippon Zenyaku Kogyo Co., Ltd.), mixed with a mixture of cell cryopreservation medium CP-1 (Kyokuto Pharmaceutical Industrial Co., Ltd.), human serum albumin (HSA) (Albuminar; CSL Behring) and RPMI, and cryopreserved at −80° C. until use. The plasma was inactivated in a water bath set at 56° C. for 30 minutes and then frozen and stored at −80° C. until use.

To activate the PBMCs, 5 μg/mL of CD3 Monoclonal Antibody (OKT3) (Thermo Fisher Scientific) and 20 μg/mL of RetroNectin (Takara Bio Inc.) that were diluted with ACD-A solution (Terumo) were added to an untreated 6-well plate at 1 mL/well. The plate was left to stand for 3 hours in an incubator set at 37° C. and 5% CO₂, and then left overnight at 4° C. to prepare an OKT3/RetroNectin-coated plate. The thawed PBMCs were suspended in a medium (GT-T551 medium (Takara Bio Inc.) supplemented with 175 IU/mL of IL-2 and 0.1% heat-inactivated autologous plasma) to prepare a cell suspension adjusted to 2.0×10⁵ cells/mL. The cell suspension was added at 6.5 mL/well to an OKT3/RetroNectin-coated plate that had been washed three times with a DMEM/F-12 medium (not containing FBS and penicillin-streptomycin). The plate was left in an incubator set at 37° C. and 5% CO₂ for 4 days to promote activation and proliferation of T cells in the PBMCs.

The RetroNectin-conjugated virus infection method was used for transduction into T cells. A RetroNectin-coated 24-well plate was prepared by the same procedure as in Example 2-(4), onto which a retroviral vector solution containing a nucleic acid encoding BCMA-CAR (L20H) derived from antibody 1 prepared in Example 2-(6) was adsorbed. To the plate, 4 mL/well of OKT3/Retronectin-activated T cells (diluted to 4.0×10⁵ cells/mL with a GT-T551 medium supplemented with 175 IU/mL of IL-2 and 0.1% heat-inactivated autologous plasma) was added. The plate was centrifuged at 32° C. and 1,960×g for 10 minutes. The plate was left overnight in an incubator set at 37° C. and 5% CO₂ to allow for transduction. The transduced T cells were collected the next day and suspended in 30 mL of a basal medium (GT-T551 medium (Takara Bio Inc.) supplemented with 0.1% heat-inactivated autologous plasma) supplemented with IL-2 at a final concentration of 175 IU/mL, and then placed in an incubator set at 37° C. and 5% CO₂ (Day 1). On days 4, 7, 9, and 11, an equal volume of the basal medium supplemented with IL-2 at a final concentration of 175 IU/mL was added. On day 14, the cells were collected and washed with CELLOTION.

### (2) Measurement of Cytotoxic Activity

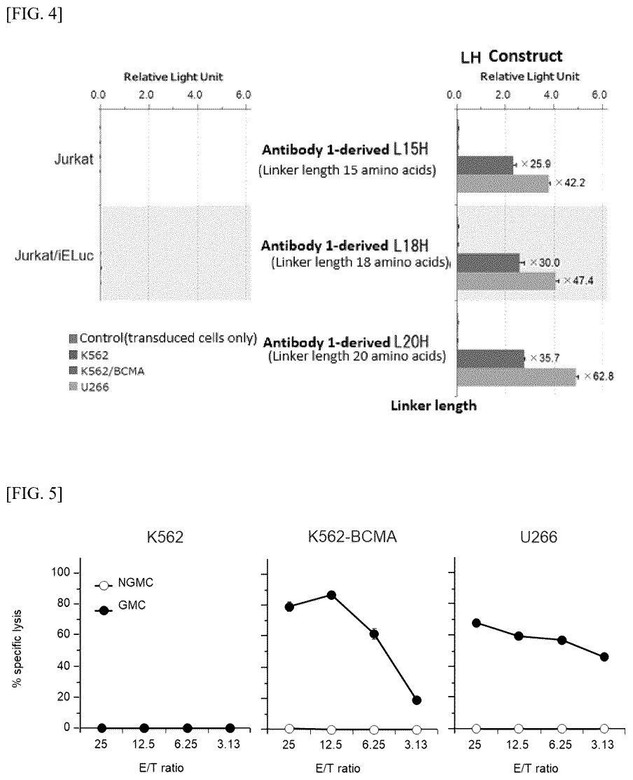

The transduced cells (GMCs) prepared in Example 3-(1) were mixed with chronic myeloid leukemia cell line K562 cells, BCMA-expressing K562 cells (K562-BCMA), or BCMA-expressing human myeloma cell line U266 cells in a V-bottom 96-well microplate at an effector:target ratio of 25:1, 12.5:1, 6.25:1, or 3.13:1, and co-cultured for 18 hours in an incubator set at 37° C. and 5% CO₂. As a control, non-transduced cells (NGMCs) were also co-cultured in the same manner. After the microplate was centrifuged at 250×g and room temperature for 10 minutes, a supernatant was dispensed into a flat-bottom 96-well microplate at 100 μL/well. The cytotoxic activity was measured using LDH Cytotoxicity Detection Kit (Takara Bio Inc.). As shown in FIG. 5, T cells expressing BCMA-CAR exhibited BCMA-specific cytotoxic activity.

### (3) Production of IFN-γ by CAR-Transduced T Cell

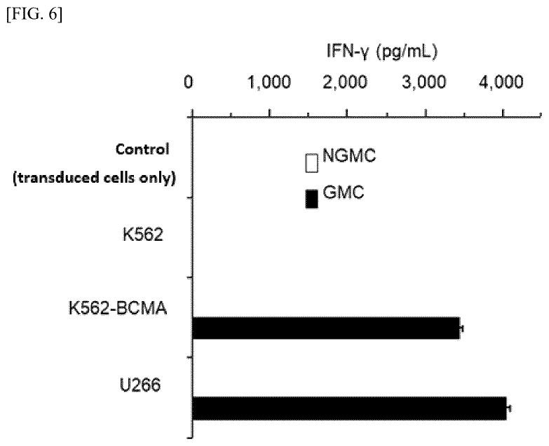

The transduced cells prepared in Example 3-(1) were mixed with K562 cells, K562-BCMA cells, or human myeloma cell line U266 cells at an effector:target ratio of 1:1 in a RPMI-1640 medium (Invitrogen) supplemented with BSA at a final concentration of 1%. The mixture was transferred to a V-bottom 96-well microplate and left to stand for 18 hours in an incubator set at 37° C. and 5% CO₂ for co-culture. The microplate was centrifuged at 250×g at room temperature for 10 minutes. A supernatant was then collected and IFN-7 production was measured using Human IFN gamma ELISA Ready-SET-Go (eBioscience). As shown in FIG. 6, T cells expressing BCMA-CAR produced IFN-7 in a BCMA-specific manner.

### (4) In Vivo Antitumor Effect of CAR-T Cell on Tumor-Bearing Mouse—1

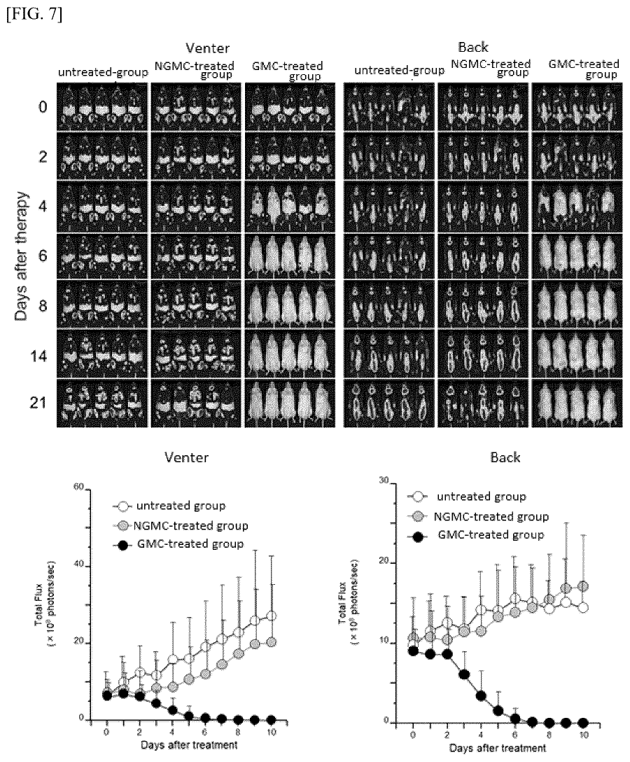

Immunodeficient NOG mice were inoculated via the tail vein with 1×10⁶ cells of human myeloma cell line U266 cells transduced with an Emerald luciferase gene, and 42 days later, the mice were transfused with 2×10⁶ cells of the CAR-transduced cells prepared in Example 3-(1) (n=10 for each). FIG. 7 shows in vivo bioluminescence imaging of luciferase activity in the tumor cells over time after infusion of each transduced T cell, and graphs quantifying the luminescence intensity. T cells expressing BCMA-CAR were found to have high antitumor activity in vivo.

### (5) In Vivo Antitumor Effect of CAR-T Cell on Tumor-Bearing Mouse—2

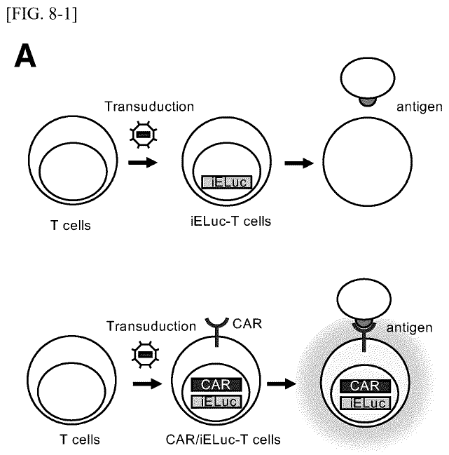

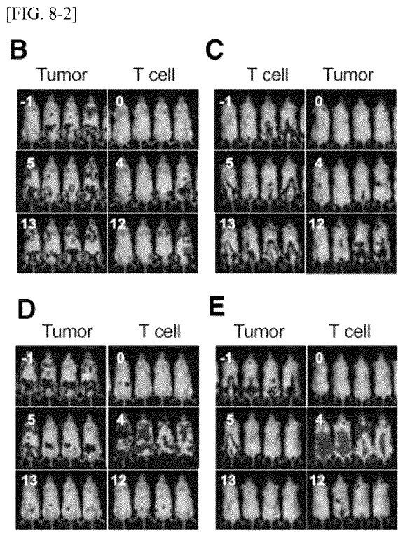

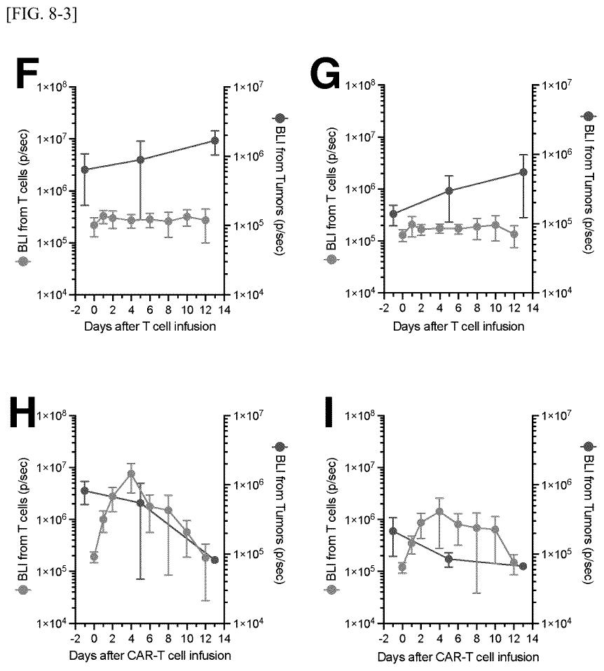

An inducible gene expression system (Uchibori R et al., Mol Ther Oncolytics 2019, 12:16-25) was used to assay the presence or absence of tumor-specific responses by infused CAR-T cells. iELuc-T refers to T cells that express luciferase in response to CAR signals. iELuc-T cells not expressing CAR (iELuc-T cell-treated group) and iELuc-T cells expressing CAR (CAR/iELuc-T cell-treated group) were infused into cancer-bearing mice in the same manner as in Example 3-(4) (FIG. 8-1, A). FIG. 8 shows in vivo bioluminescence imaging of the luciferase activity in the tumor cells over time after infusion in the groups and the luciferase activity in response to CAR signals, as well as graphs quantifying the luminescence intensity. In the iELuc-T cell-treated group, though partial activation of the infused cells was observed., the tumor cells continued to grow thereafter (FIGS. 8-2 and 8-3, B, C, F, and G). In contrast, in the CAR/iELuc-T cell-treated group, systemic activation of the infused cells was observed, resulting in the disappearance of tumor cells (FIGS. 8-2 and 8-3, D, E, H, and I). It was found that the infused CAR-T cells exhibited tumor cell-specific cytotoxic responses. Interestingly, the activation of the infused T cells rapidly decreased with the disappearance of the tumor cells.

### (6) Inhibitory Effect on the Progression of Anemia

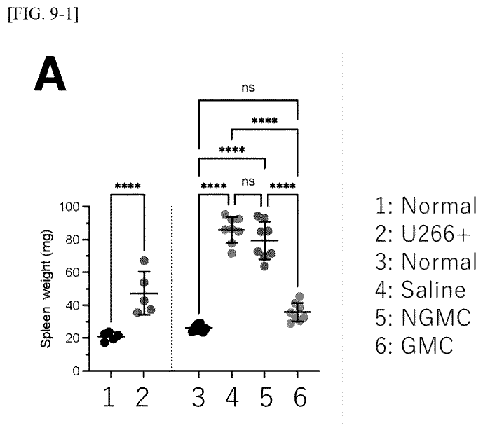

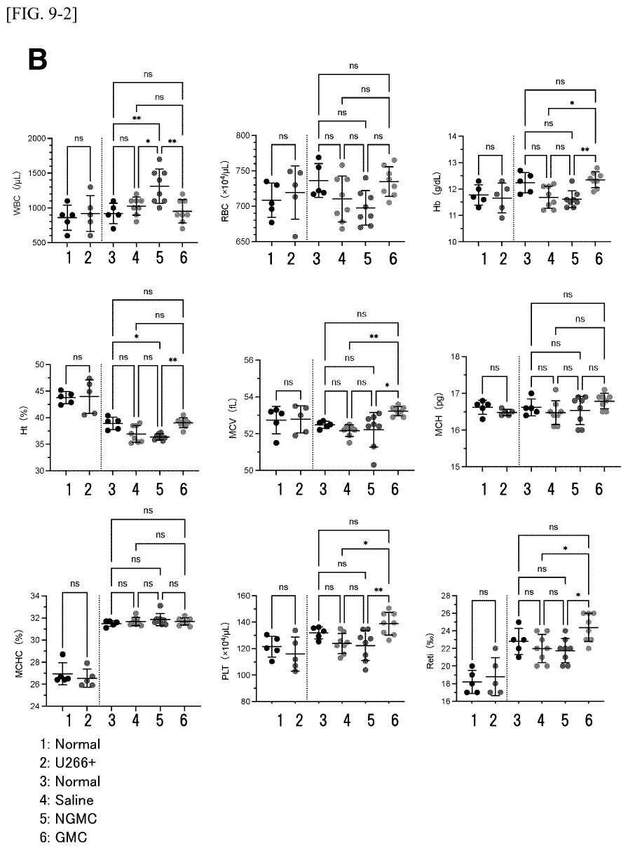

Erythropoiesis Occurs in Structural and Functional Units of the Bone Marrow, Called erythroblastic islands (EBIs). After partial maturation in the bone marrow, reticulocytes enter the blood where they become mature red blood cells. Myeloma cells infiltrating the bone marrow damage the function and structure of EBIs by secreting cytokines. The total number of macrophages in the bone marrow of MM patients correlates inversely with the area of bone marrow infiltration by myeloma cells. Although EBI central macrophages represent a small proportion of total bone marrow macrophages, a proportional reduction in the bone marrow central macrophage population leads to a decline in the number of EBIs and the erythropoietic activity. It was found that the spleen weight of the cancer-bearing mice (U266+) was significantly increased as compared to that of the normal mice (Normal). This is probably because infiltration of U266 cells into the bone marrow inhibited normal intramedullary hematopoiesis, resulting in enhanced extramedullary hematopoiesis in the spleen. The spleen weight also decreased in the GMC-treated group, though it was higher than Normal, and returned to normal weight (FIG. 9-1, A).

Furthermore, fetal hepatic hematopoiesis in mice disappears approximately two weeks after birth. However, during acute experimental anemia, extramedullary hematopoiesis occurs in the liver of adult mice. Therefore, it is expected that it is technically difficult to reproduce the anemic pathology of MM patients in mice. Hematological parameters before treatment and 21 days after treatment are shown in FIG. 9B. In FIG. 9B, WBC represents white blood cells, RBC represents red blood cells, Hb represents hemoglobin, Hf represents hematocrit, MCV represents mean corpuscular volume, MCH represents mean corpuscular hemoglobin, MCHC represents mean corpuscular hemoglobin concentration, PLT represents platelet count, and Reti represents reticulocyte count. In this study, though tumor-bearing mice showed a significant decrease in MCH at the start of treatment, the numbers of RBCs and reticulocytes tended to increase. However, mice treated with saline and NGMC tended to become anemic over time, with decreased red blood cells and reticulocytes. In contrast, in mice treated with GMC, the progression of anemia was prevented and was similar to that observed in normal mice (FIG. 9-2, B).

### (7) Inhibitory Effect of CAR-T Therapy on Progression in Bone Areas

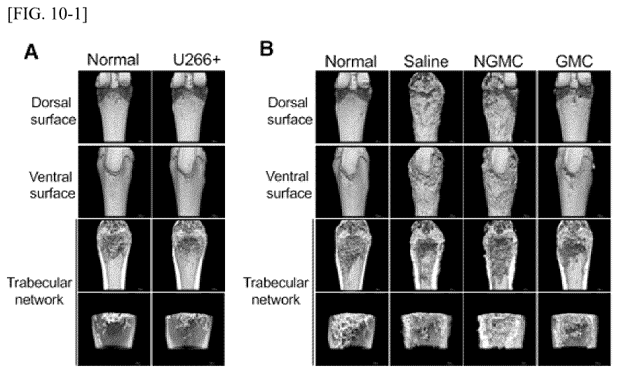

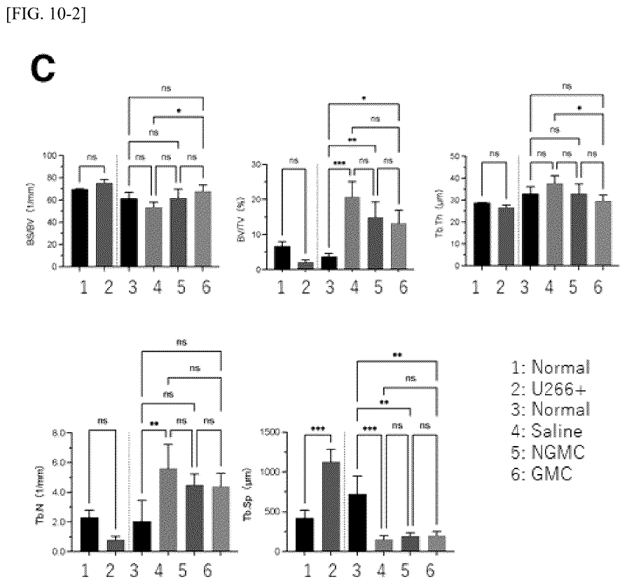

The morphology of bone trabeculae was evaluated using micro-CT (FIF. 10-1 and 10-2). In FIG. 10-2, BS/BV represents a ratio of bone surface area/volume, BS/TV represents the bone volume per tissue volume, Tb.Th represents the trabecular width, Tb.N represents the trabecular number, and Tb.Sp represents the trabecular center distance.

At the start of treatment, though tumor-bearing mice showed slightly advanced bone lesions as compared to normal mice, the bone surface of the tumor-bearing mice was similar to that of normal mice (FIGS. 10-1 and 10-2, A and C). Mice treated with saline or NGMC showed progressive bone lesions. Severe destruction of the condyle and bone surface was observed (FIG. 10-1 and FIG. 10-2, B, C). In contrast, in mice treated with GMC, the progression of bone lesions was strongly prevented, and the bone surface was similar to that of normal mice. Because the observation period was short, it is thought that the phenomenon observed in this study was not an improvement in bone lesions caused by GMC administration, but rather a reduction in progression.

### INDUSTRIAL APPLICABILITY

The present invention provides a CAR useful in the field of gene-modified immune cell therapy targeting BCMA, a nucleic acid encoding the CAR, a vector containing the nucleic acid, a cell expressing the chimeric receptor, a method for producing the cell, a pharmaceutical composition containing the cell as an active ingredient, and a method for preventing (including prevention of recurrence; the same applies hereinafter) or treating a disease, the method comprising administering the pharmaceutical composition to a subject. The cells expressing the CAR of the present invention have high killing ability against cells expressing BCMA, and are expected to have a high therapeutic effect.

1. An anti-human B-cell maturation antigen (BCMA) antibody or an antigen-binding fragment thereof, comprising a light chain variable (VL) region and a heavy chain variable (VH) region, wherein the amino acid sequences of complementarity-determining regions (CDR) 1, CDR2, and CDR3 of the VL region and CDR1, CDR2, and CDR3 of the VH region are amino acid sequences set forth in SEQ ID NOs selected from the group consisting of the following (1) to (6):
(1) SEQ ID NOs: 1 to 6,
(2) SEQ ID NOs: 7 to 12,
(3) SEQ ID NOs: 13 to 18,
(4) SEQ ID NOs: 19 to 24,
(5) SEQ ID NOs: 25 to 30, and
(6) SEQ ID NOs: 31 to 36.

2. The antibody or antigen-binding fragment thereof according to claim 1, wherein the amino acid sequences of the VL and VH regions are selected from the group consisting of the following (1) to (6):
(1) SEQ ID NOs: 37 and 38,
(2) SEQ ID NOs: 39 and 40,
(3) SEQ ID NOs: 41 and 42,
(4) SEQ ID NOs: 43 and 44,
(5) SEQ ID NOs: 45 and 46, and
(6) SEQ ID NOs: 47 and 48.

3. The antibody or antigen-binding fragment thereof according to claim 1, wherein the antibody or antigen-binding fragment thereof is selected from the group consisting of IgG, Fab, Fab′, F(ab′)2, Fv, scFv, and single domain antibody (dAB).

4. The antigen-binding fragment according to claim 3, wherein the antigen-binding fragment is an scFv.

5. A nucleic acid encoding the antibody or antigen-binding fragment thereof according to claim 1.

6. A chimeric antigen receptor (CAR) comprising (i) an antigen-binding domain, (ii) a transmembrane domain, and (iii) an intracellular domain, wherein the antigen-binding domain comprises the antigen-binding fragment according to claim 1.

7. The CAR according to claim 6, wherein the intracellular domain comprises a costimulatory domain and an activation domain.

8. The CAR according to claim 7, wherein the costimulatory domain comprises an intracellular fragment of CD28.

9. The CAR according to claim 7, wherein the activation domain comprises an intracellular fragment of CD3ζ.

10. A nucleic acid encoding the CAR according to claim 6.

11. A vector comprising the nucleic acid encoding the antibody or antigen-binding fragment thereof according to claim 5.

12. A cell comprising the nucleic acid encoding the antibody or antigen-binding fragment thereof according to claim 5.

13. The cell according to claim 12, wherein the cell is a T cell.

14. A pharmaceutical composition comprising the cells according to claim 13 as an active ingredient.

15. A method for producing a cell expressing a CAR, the method comprising transducing a cell with the nucleic acid encoding the antibody or antigen-binding fragment thereof according to claim 5.

16. A nucleic acid encoding the antibody or antigen-binding fragment thereof according to claim 2.

17. A chimeric antigen receptor (CAR) comprising (i) an antigen-binding domain, (ii) a transmembrane domain, and (iii) an intracellular domain, wherein the antigen-binding domain comprises the antigen-binding fragment according to claim 2.

18. A nucleic acid encoding the CAR according to claim 17.

19. A vector comprising the nucleic acid encoding the antibody or antigen-binding fragment thereof according to claim 18.

20. A method for producing a cell expressing a CAR, the method comprising transducing a cell with the nucleic acid encoding the antibody or antigen-binding fragment thereof according to claim 16.


SEQUENCE LISTING (70 sequences)

SEQ ID NO: 1 - protein, 15 aa, Mus musculus
  source 1..15: note = Anti-BCMA Antibody 1 VL CDR1
RASKSVSTSGYSYMH

SEQ ID NO: 2 - protein, 7 aa, Mus musculus
  source 1..7: note = Anti-BCMA Antibody 1 VL CDR2
LASNLES

SEQ ID NO: 3 - protein, 9 aa, Mus musculus
  source 1..9: note = Anti-BCMA Antibody 1 VL CDR3
QHSRELPFT

SEQ ID NO: 4 - protein, 5 aa, Mus musculus
  source 1..5: note = Anti-BCMA Antibody 1 VH CDR1
SYVMH

SEQ ID NO: 5 - protein, 17 aa, Mus musculus
  source 1..17: note = Anti-BCMA Antibody 1 VH CDR2
YIIPYNDGTKYNEKFKG

SEQ ID NO: 6 - protein, 12 aa, Mus musculus
  source 1..12: note = Anti-BCMA Antibody 1 VH CDR3
LTFSYDSYAMDF

SEQ ID NO: 7 - protein, 15 aa, Mus musculus
  source 1..15: note = Anti-BCMA Antibody 2 VL CDR1
RASKSVSTSGYSYMH

SEQ ID NO: 8 - protein, 7 aa, Mus musculus
  source 1..7: note = Anti-BCMA Antibody 2 VL CDR2
LASNLES

SEQ ID NO: 9 - protein, 9 aa, Mus musculus
  source 1..9: note = Anti-BCMA Antibody 2 VL CDR3
QHIRELPIT

SEQ ID NO: 10 - protein, 5 aa, Mus musculus
  source 1..5: note = Anti-BCMA Antibody 2 VH CDR1
NYVMH

SEQ ID NO: 11 - protein, 17 aa, Mus musculus
  source 1..17: note = Anti-BCMA Antibody 2 VH CDR2
YIIPYNDGTKYNEKFKG

SEQ ID NO: 12 - protein, 12 aa, Mus musculus
  source 1..12: note = Anti-BCMA Antibody 2 VH CDR3
LTFSYDSYAMDY

SEQ ID NO: 13 - protein, 15 aa, Mus musculus
  source 1..15: note = Anti-BCMA Antibody 3 VL CDR1
RASKNVSTSGYRYMH

SEQ ID NO: 14 - protein, 7 aa, Mus musculus
  source 1..7: note = Anti-BCMA Antibody 3 VL CDR2
LASNLES

SEQ ID NO: 15 - protein, 9 aa, Mus musculus
  source 1..9: note = Anti-BCMA Antibody 3 VL CDR3
QHSRELPFT

SEQ ID NO: 16 - protein, 6 aa, Mus musculus
  source 1..6: note = Anti-BCMA Antibody 3VH CDR1
SYIMHW

SEQ ID NO: 17 - protein, 17 aa, Mus musculus
  source 1..17: note = Anti-BCMA Antibody 3 VH CDR2
YIIPYNDGTKYNEKFKG

SEQ ID NO: 18 - protein, 12 aa, Mus musculus
  source 1..12: note = Anti-BCMA Antibody 3 VH CDR3
LTFSYDSYAMDY

SEQ ID NO: 19 - protein, 15 aa, Mus musculus
  source 1..15: note = Anti-BCMA Antibody 4 VL CDR1
RASKSVSTSGYSYMH

SEQ ID NO: 20 - protein, 7 aa, Mus musculus
  source 1..7: note = Anti-BCMA Antibody 4 VL CDR2
LASNLES

SEQ ID NO: 21 - protein, 9 aa, Mus musculus
  source 1..9: note = Anti-BCMA Antibody 4 VL CDR3
QHSRELPFT

SEQ ID NO: 22 - protein, 6 aa, Mus musculus
  source 1..6: note = Anti-BCMA Antibody 4 VH CDR1
NYIIHW

SEQ ID NO: 23 - protein, 17 aa, Mus musculus
  source 1..17: note = Anti-BCMA Antibody 4 VH CDR2
YIIPYNDGSKYNEKFKG

SEQ ID NO: 24 - protein, 12 aa, Mus musculus
  source 1..12: note = Anti-BCMA Antibody 4 VH CDR3
LTFSYDSYAMDY

SEQ ID NO: 25 - protein, 15 aa, Mus musculus
  source 1..15: note = Anti-BCMA Antibody 5 VL CDR1
RASKSVSTSGYSYIH

SEQ ID NO: 26 - protein, 7 aa, Mus musculus
  source 1..7: note = Anti-BCMA Antibody 5 VL CDR2
LASNLES

SEQ ID NO: 27 - protein, 9 aa, Mus musculus
  source 1..9: note = Anti-BCMA Antibody 5 VL CDR3
QHSRELPFT

SEQ ID NO: 28 - protein, 6 aa, Mus musculus
  source 1..6: note = Anti-BCMA Antibody 5 VH CDR1
NYIMHW

SEQ ID NO: 29 - protein, 17 aa, Mus musculus
  source 1..17: note = Anti-BCMA Antibody 5 VH CDR2
YIIPYNDGTKYNEQFTG

SEQ ID NO: 30 - protein, 12 aa, Mus musculus
  source 1..12: note = Anti-BCMA Antibody 5 VH CDR3
LTFSYDSYAMDY

SEQ ID NO: 31 - protein, 15 aa, Mus musculus
  source 1..15: note = Anti-BCMA Antibody 6 VL CDR1
RASKSVSTSGYSYMH

SEQ ID NO: 32 - protein, 7 aa, Mus musculus
  source 1..7: note = Anti-BCMA Antibody 6 VL CDR2
LASNLES

SEQ ID NO: 33 - protein, 9 aa, Mus musculus
QHSRELPFT

SEQ ID NO: 34 - protein, 6 aa, Mus musculus
GYIMHW

SEQ ID NO: 35 - protein, 17 aa, Mus musculus
  source 1..17: note = Anti-BCMA Antibody 6 VH CDR2
YIIPYNDGTKYNEKFKG

SEQ ID NO: 36 - protein, 12 aa, Mus musculus
  source 1..12: note = Anti-BCMA Antibody 6 VH CDR3
LTFSYDSYAMDY

SEQ ID NO: 37 - protein, 111 aa, Mus musculus
  source 1..111: note = Anti-BCMA Antibody 1 VL
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPFTFGSGTKLEIK

SEQ ID NO: 38 - protein, 121 aa, Mus musculus
  source 1..121: note = Anti-BCMA Antibody 1 VH
EVQLQQSGPELVKPGASVKMSCKASGYTYTSYVMHWVKQKPGQGLEWIGYIIPYNDGTKYNEKFKGKATLTSDKSSSTAYMELSSLTSEDSAVYYCARLTFSYDSYAMDFWGQGTSVTVSS

SEQ ID NO: 39 - protein, 111 aa, Mus musculus
  source 1..111: note = Anti-BCMA Antibody 2 VL
DIVLTQSPASLAVSLGQRATISCRASKSVSTSGYSYMHWYQQKPGQPPKLLIYLASNLESGVPARFSGSGSGTDFTLNIHPVEEEDAATYYCQHIRELPITFGSGTKLEIK

SEQ ID NO: 40 - protein, 121 aa, Mus musculus
  source 1..121: note = Anti-BCMA Antibody 2 VH
EVQLQQSGPELVKPGASVKMSCKASGYTFTNYVMHWVKQKPGQGLEWIGYIIPYNDGTKYNEKFKGKATLTSDKSSSTAYMELTSLTSEDSAVFYCARLTFSYDSYAMDYWGQGTSVTVSS

SEQ ID NO: 41 - protein, 111 aa, Mus musculus
  source 1..111: note = Anti-BCMA Antibody 3 VL
DIVLTQSPASLAVSLGQRATISCRASKNVSTSGYRYMHWYQQKSGQPPKLLIYLASNLESGVPARFSGSGSGTDFTLNIHPVEEEDAATYYCQHSRELPFTFGSGTKLEIK

SEQ ID NO: 42 - protein, 121 aa, Mus musculus
  source 1..121: note = Anti-BCMA Antibody 3 VH
EVQLQQSGPELVKPGASVKMSCKASGYTFTSYIMHWVKQKPGQGLEWIGYIIPYNDGTKYNEKFKGKATLTSDKSSSTAYMELSSLTSEDSAVYYCARLTFSYDSYAMDYWGQGTSVTVSS

SEQ ID NO: 43 - protein, 111 aa, Mus musculus
  source 1..111: note = Anti-BCMA Antibody 4 VL
DIVLTQSPASLAVSLGQRATISCRASKSVSTSGYSYMHWYQQKPGQPPRLLIYLASNLESGVPARFSGSGSGTDFTLNIHPVEEEDAATYYCQHSRELPFTFGSGTKLEIK

SEQ ID NO: 44 - protein, 121 aa, Mus musculus
  source 1..121: note = Anti-BCMA Antibody 4 VH
EVQLQQSGPELVKPGASVKMSCKASGYTFTNYIIHWVKQKPGQGLEWIGYIIPYNDGSKYNEKFKGKATLTSDKSSSTAYMELSSLTSEDSAVYYCARLTFSYDSYAMDYWGQGTSVTVSS

SEQ ID NO: 45 - protein, 111 aa, Mus musculus
  source 1..111: note = Anti-BCMA Antibody 5 VL
DIVLTQSPASLAVSLGQRATISCRASKSVSTSGYSYIHWYQQKPGQPPKLLIYLASNLESGVPARFSGSGSGTDFTLNIHPVEEEDAATYYCQHSRELPFTFGSGTKLEIK

SEQ ID NO: 46 - protein, 121 aa, Mus musculus
  source 1..121: note = Anti-BCMA Antibody 5 VH
EVQLQQSGPELVKPGASVKMSCKASGYTFTNYIMHWVKQRPGQGLEWIGYIIPYNDGTKYNEQFTGKATLTSDKSSSTAYMELSSLTSEDSAVYYCARLTFSYDSYAMDYWGQGTSVTVSS

SEQ ID NO: 47 - protein, 111 aa, Mus musculus; identical to SEQ ID NO: 37
  source 1..111: note = Anti-BCMA Antibody 6 VL

SEQ ID NO: 48 - protein, 121 aa, Mus musculus
  source 1..121: note = Anti-BCMA Antibody 6 VH
EVQLQQSGPELVKPGASVKMSCKASGYTFTGYIMHWVKQKPGQGLEWIGYIIPYNDGTKYNEKFKGKATLTSDKSSSTAYMELSSLTSEDSAVYYCARLTFSYDSYAMDYWGQGTSVTVSS

SEQ ID NO: 49 - DNA, 333 bp, Mus musculus
  source 1..333: mol_type = genomic DNA; note = Anti-BCMA Antibody 1 VL
gacattgtgctgacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccattcacgttcggctcggggacaaagttggaaataaaa

SEQ ID NO: 50 - DNA, 363 bp, Mus musculus
  source 1..363: mol_type = genomic DNA; note = Anti-BCMA Antibody 1 VH
gaggtccagctgcagcagtctggacctgagctggtaaagcctggggcttcagtgaagatgtcctgcaaggcttctggatacacatacactagctatgttatgcactgggtgaagcagaagcctgggcagggccttgagtggattggatatattattccttacaatgatggtactaagtacaatgagaagttcaagggcaaggccacactgacttcagacaaatcctccagcacagcctacatggagctcagcagcctgacctctgaggactctgcggtctattactgtgcaagattgaccttttcttacgactcctatgctatggacttctggggtcaaggaacctcagtcaccgtctcctca

SEQ ID NO: 51 - DNA, 333 bp, Mus musculus
  source 1..333: mol_type = genomic DNA; note = Anti-BCMA Antibody 2 VL
gacattgtgctgacacagtctcctgcttccttagctgtatctctggggcagagggccaccatctcatgcagggccagcaaaagtgtcagtacatctggttatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacgtattactgtcagcacattagggagcttccaatcacgttcggctcggggacaaagttggaaataaaa

SEQ ID NO: 52 - DNA, 363 bp, Mus musculus
  source 1..363: mol_type = genomic DNA; note = Anti-BCMA Antibody 2 VH
gaggtccagctgcagcagtctggacctgagctggtaaagcctggggcttcagtgaagatgtcctgcaaggcttctggatacacattcactaactatgttatgcactgggtgaagcagaagcctgggcagggccttgagtggattggatatattattccttataatgatggtactaaatacaatgagaagttcaaaggcaaggccacactgacttcagacaaatcctccagcacagcctacatggagctcaccagcctgacctctgaggactctgcggtcttttactgtgcaagattgaccttttcttacgactcctatgctatggactactggggtcaaggaacctcagtcaccgtctcctca

SEQ ID NO: 53 - DNA, 333 bp, Mus musculus
  source 1..333: mol_type = genomic DNA; note = Anti-BCMA Antibody 3 VL
gacattgtgctgacacagtctcctgcttccttagctgtatctctggggcagagggccaccatctcatgcagggccagcaaaaatgtcagtacatctggctataggtacatgcactggtaccaacagaaatcaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccattcacgttcggctcggggacaaagttggaaataaaa

SEQ ID NO: 54 - DNA, 363 bp, Mus musculus
  source 1..363: mol_type = genomic DNA; note = Anti-BCMA Antibody 3 VH
gaggtccagctgcagcagtctggacctgagctggtaaagcctggggcttcagtgaagatgtcctgcaaggcttctggatacacattcactagctatattatgcactgggtgaagcagaagcctgggcagggccttgagtggattggatatattattccttacaatgatggtactaagtacaatgagaagttcaaaggcaaggccacactgacttcagacaaatcctccagcacagcctacatggagctcagcagcctgacctctgaggactctgcggtctattactgtgcaagattgaccttttcttacgactcctatgctatggactactggggtcaaggaacctcagtcaccgtctcctca

SEQ ID NO: 55 - DNA, 333 bp, Mus musculus
  source 1..333: mol_type = genomic DNA; note = Anti-BCMA Antibody 4 VL
gacattgtgctgacacagtctcctgcttccttagctgtatctctggggcagagggccaccatctcatgcagggccagcaaaagtgtcagtacatctggctatagttatatgcactggtaccaacagaaaccaggacagccacccagactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccattcacgttcggctcggggacaaagttggaaataaaa

SEQ ID NO: 56 - DNA, 363 bp, Mus musculus
  source 1..363: mol_type = genomic DNA; note = Anti-BCMA Antibody 4 VH
gaggtccagctgcagcagtctggacctgagctggtaaagcctggggcttcagtgaagatgtcctgcaaggcttctggatacacattcactaactatattatacactgggtgaagcagaagcctgggcagggccttgagtggattggatatattattccttacaatgatggttctaagtacaatgagaagttcaaaggcaaggccacactgacttcagacaaatcctccagcacagcctacatggagctcagcagcctgacctctgaggactctgcggtctattactgtgcaagattgaccttttcttacgactcctatgctatggactactggggtcaaggaacctcagtcaccgtctcctca

SEQ ID NO: 57 - DNA, 333 bp, Mus musculus
  source 1..333: mol_type = genomic DNA; note = Anti-BCMA Antibody 5 VL
gacattgtgctgacacagtctcctgcttccttagctgtatctctggggcagagggccaccatctcatgcagggccagcaaaagtgtcagtacatctggctatagttatatacactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccattcacgttcggctcggggacaaagttggaaataaaa

SEQ ID NO: 58 - DNA, 363 bp, Mus musculus
  source 1..363: mol_type = genomic DNA; note = Anti-BCMA Antibody 5 VH
gaggtccagctgcagcagtctggacctgagctggtaaagcctggggcttcagtgaagatgtcctgcaaggcttctggatacacattcactaactatattatgcactgggtgaagcagaggcctgggcagggccttgagtggattggatatattattccttacaatgatggtactaagtacaatgagcagttcacaggcaaggccacactgacttcagacaaatcctccagcacagcctacatggagctcagcagcctgacctctgaggactctgcggtctattactgtgcaagattgaccttttcttacgactcctatgctatggactactggggtcaaggaacctcagtcaccgtctcctca

SEQ ID NO: 59 - DNA, 333 bp, Mus musculus; identical to SEQ ID NO: 49
  source 1..333: mol_type = genomic DNA; note = Anti-BCMA Antibody 6 VL

SEQ ID NO: 60 - DNA, 363 bp, Mus musculus
  source 1..363: mol_type = genomic DNA; note = Anti-BCMA Antibody 6 VH
gaggtccagctgcagcagtctggacctgagctggtaaaacctggggcttctgtgaagatgtcctgcaaggcttctggatacacattcactggctatattatgcactgggtgaagcagaagcctgggcagggccttgagtggattggatatattattccttacaatgatggtactaagtacaatgagaagttcaaaggcaaggccacactgacttcagacaaatcctccagcacagcctacatggagctcagcagcctgacctctgaggactctgcggtctattactgtgcaagattgaccttttcttacgactcctatgctatggactactggggtcaaggaacctcagtcaccgtctcctca

SEQ ID NO: 61 - protein, 12 aa, Synthetic construct
GGGSGGGSGGGS

SEQ ID NO: 62 - protein, 15 aa, Synthetic construct
GGGGSGGGGSGGGGS

SEQ ID NO: 63 - protein, 20 aa, Synthetic construct
GGGGSGGGGSGGGGSGGGGS

SEQ ID NO: 64 - protein, 18 aa, Synthetic construct
GGSSRSSSSGGGGSGGGG

SEQ ID NO: 65 - protein, 499 aa, Synthetic construct
  source 1..499: note = BCMA-CAR(L20H)
MLLLVTSLLLCELPHPAFLLIPDIVLTQSPASLAVSLGQRATISCRASKSVSTSGYSYMHWYQQKPGQPPRLLIYLASNLESGVPARFSGSGSGTDFTLNIHPVEEEDAATYYCQHSRELPFTFGSGTKLEIKGGGGSGGGGSGGGGSGGGGSEVQLQQSGPELVKPGASVKMSCKASGYTFTNYIIHWVKQKPGQGLEWIGYIIPYNDGSKYNEKFKGKATLTSDKSSSTAYMELSSLTSEDSAVYYCARLTFSYDSYAMDYWGQGTSVTVSSATGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - DNA, 1500 bp, Synthetic construct
  source 1..1500: mol_type = other DNA; note = BCMA-CAR(L20H)
atgctgctcctggtgaccagcttgctgctgtgcgagcttcctcacccagccttcctcctgatccccgacattgtgctgacacagtctcctgcttccttagctgtatctctggggcagagggccaccatctcatgcagggccagcaaaagtgtcagtacatctggctatagttatatgcactggtaccaacagaaaccaggacagccacccagactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccattcacgttcggctcggggacaaagttggaaataaaagggggaggaggatctggtggaggagggagtggcggtggcggatctggaggtggaggaagcgaggtccagctgcagcagtctggacctgagctggtaaagcctggggcttcagtgaagatgtcctgcaaggcttctggatacacattcactaactatattatacactgggtgaagcagaagcctgggcagggccttgagtggattggatatattattccttacaatgatggttctaagtacaatgagaagttcaaaggcaaggccacactgacttcagacaaatcctccagcacagcctacatggagctcagcagcctgacctctgaggactctgcggtctattactgtgcaagattgaccttttcttacgactcctatgctatggactactggggtcaaggaacctcagtcaccgtctcctcagctaccggtgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 67 - DNA, 42 bp, Synthetic construct
  source 1..42: mol_type = other DNA
gattacgccaagcttctcattcctgttgaagctcttgacaat

SEQ ID NO: 68 - DNA, 41 bp, Synthetic construct
  source 1..41: mol_type = other DNA
gattacgccaagcttctcaattttcttgtccaccttggtgc

SEQ ID NO: 69 - DNA, 17 bp, Synthetic construct
  source 1..17: mol_type = other DNA
caggaaacagctatgac

SEQ ID NO: 70 - DNA, 17 bp, Synthetic construct
  source 1..17: mol_type = other DNA
gtaaaacgacggccagt